(19) United States
(12) Patent Application Publication
(10) Pub. No.: US 2025/0017666 A1
(43) Pub. Date: Jan. 16, 2025

(54) Surgical Tracker Assemblies With Optimized Size, Weight, And Accuracy

(71) Applicant: MAKO Surgical Corp., Weston, FL (US)

(72) Inventors: Matthew Joseph Guthart, Miami, FL (US); Mark Ellsworth Nadzadi, Batavia, OH (US); Koustubh Rao, Hallandale Beach, FL (US); Andrew Jacob Nelson, New York City, NY (US); Vishal Dilip Jagtap, Thane (IN)

(73) Assignee: MAKO Surgical Corp., Weston, FL (US)

(21) Appl. No.: 18/768,405

(22) Filed: Jul. 10, 2024

Related U.S. Application Data
(60) Provisional application No. 63/526,733, filed on Jul. 14, 2023.

(51) Int. Cl. A61B 34/20

(52) U.S. Cl. CPC A61B 34/20; A61B 2034/2072

(57) ABSTRACT

A surgical tracker assembly includes a tracker body including a first connector. The surgical tracker assembly also includes a mount assembly couplable to the tracker body. The mount assembly includes a second connector engageable with the first connector in at least a first position and a second position different from the first position. One of the first connector and the second connector includes a multipositional receptacle extending between a first receptacle end and a second receptacle end. The first receptacle end defines a first opening, and the second receptacle end defines a second opening. The other of the first connector and the second connector includes a projection disposable in the first opening to couple the tracker body to the mount assembly in the first position and disposable in the second opening to couple to the tracker body to the mount assembly in the second position.

### CROSS-REFERENCE TO RELATED APPLICATIONS

The subject patent application claims priority to and all the benefits of U.S. Provisional Patent Application No. 63/526,733, which was filed on Jul. 14, 2023, which is herein incorporated by reference in its entirety.

### BACKGROUND

Surgical trackers known in the art are used to track objects, such as surgical tools and the patient, during a surgical procedure. Often, these trackers include fiducials which are viewable by a localizer to determine the position of the tracker, and thus determine the position of the object being tracked. Conventional trackers suffer from various design difficulties. For example, conventional trackers often have a large footprint in order to space the fiducials far apart from one other to achieve a required accuracy. In turn, the large footprint interferes with the surgical site and potentially cause unwanted collisions. Conventional trackers are also heavy because the tracking markers are supported by a metallic body with multiple arms. The mass of these trackers can cause issues such as instable installation of tracker to the object and fatigue to a surgeon who may need to support the tracker while using the tracked object. Increasing the number of tracking markers can increase tracking accuracy by the localizer. However, adding more markers to these conventional trackers will exacerbate the issues described above. Furthermore, many conventional trackers must be manufactured in a manner that causes tolerance stack, which can result in inaccuracies. Furthermore, some conventional trackers must be hermetically sealed to protect internal components of the tracker. Hermetically sealing the tracker adds complications and cost to the design and exposes the tracker to the risk of seal breaking. For many of the reasons above, conventional trackers are also not disposable or suitable for single use. Other prior trackers require multiple support components to create a rigid connection between the tracker and the object. These multiple support components increase complexity, weight, and footprint of the design. Furthermore, many conventional trackers are connectable to the support components in only one way. Other trackers can be manually adjusted relative to the support components but require a significant number of steps to perform the adjustment.

### SUMMARY

This Summary introduces a selection of concepts in a simplified form that are further described below in the Detailed Description below. This Summary is not intended to limit the scope of the claimed subject matter nor identify key features or essential features of the claimed subject matter.

In a first aspect, a surgical tracker assembly for tracking an object is provided. The surgical tracker assembly includes a tracker body including a first connector and a mount assembly couplable to the tracker body. The mount assembly includes a second connector engageable with the first connector in at least a first position and a second position different from the first position. One of the first connector and the second connector includes a multipositional receptacle. The multipositional receptacle extends between a first receptacle end and a second receptacle end. The first receptacle end defines a first opening and the second receptacle end defines a second opening. The other of the first connector and the second connector includes a projection. The projection is disposable in the first opening of the first receptacle end to couple the tracker body to the mount assembly in the first position, and the projection is disposable in the second opening of the second receptacle end to couple the tracker body to the mount assembly in the second position.

In a second aspect, the surgical tracker assembly for tracking an object includes a tracker body and a mount assembly. The mount assembly includes a first attachment portion connectable to the tracker body and a second attachment portion connectable to the object. The first attachment portion defines a first keyed geometry and includes flexible connection members. The second attachment portion defines an aperture configured to receive and connect with the flexible connection members of the first attachment portion through either side of the aperture. The aperture defined by the second keyed geometry conforms to the first keyed geometry such that the second attachment portion receives the first attachment portion in a predefined orientation.

In a third aspect, the surgical tracker assembly includes a tracker body having a back surface. The back surface defines an attachment base. The surgical tracker assembly also includes a first attachment mechanism including a first keying feature including flexures and tabs. The first keying feature has a shape of irregular geometry. The surgical tracker assembly further includes a second attachment mechanism including an open ring including a second keying feature which is configured to receive the first keying feature. The open ring has a shape of irregular geometry complementary to the shape of the first keying feature. The second attachment mechanism is configured to receive the first attachment mechanism through either side of the open ring and maintain a snap lock between the first attachment mechanism and the second attachment mechanism. The attachment base is rigidly coupled to one of the first attachment mechanism and the second attachment mechanism.

In a fourth aspect, a mount assembly for a surgical tracker is provided. The mount assembly includes a first attachment base connected to a tracker body, a second attachment base connected to an object, a first attachment mechanism including a first keying feature including flexures and tabs, and a second attachment mechanism including an open ring. The open ring includes a second keying feature which is configured to receive the first keying feature. The second attachment mechanism is configured to receive the first attachment mechanism through either side of the open ring. The first attachment base is rigidly coupled to one of the first attachment mechanism and the second attachment mechanism. The second attachment base is coupled to the other of the first attachment mechanism and the second attachment mechanism.

In a fifth aspect, the surgical tracker assembly for tracking an object includes a tracker body and a mount assembly. The mount assembly includes a first attachment portion coupled to the tracker body and including a connection member. The mount assembly also includes a second attachment portion couplable to the object, the second attachment portion includes a receptacle including an open ring configured to receive the connection member therethrough. The receptacle is configured to receive and connect with the connection member through either one of opposing sides of the open ring.

In a sixth aspect, a mount assembly for a surgical tracker assembly configured to track an object is provided. The mount assembly includes a first attachment portion coupled or connectable to a tracker body. The first attachment portion includes a connection member. The mount assembly also includes a second attachment portion coupled or connectable to the object. The second attachment portion includes a receptacle including an open ring configured to receive the connection member therethrough. The receptacle is configured to receive and connect with the connection member through either one of opposing sides of the open ring.

In a seventh aspect, the surgical tracker assembly for tracking an object includes a tracker body and a mount assembly. The mount assembly includes a first attachment portion coupled or connectable to the object. The first attachment portion including a first connection member extending in a first direction and a second connection member extending in a second direction. The mount assembly also includes a second attachment portion coupled or connectable to the tracker body. The second attachment portion includes a receptacle including an open ring. The receptacle is configured to receive and connect with either of the first or the second connection member through either one of opposing sides of the open ring.

In an eighth aspect, a mount assembly for a surgical tracker assembly configured to track an object is provided. The mount assembly includes a first attachment portion coupled or connectable to the object. The first attachment portion includes a first connection member extending in a first direction and a second connection member extending in a second direction. The mount assembly also includes a second attachment portion coupled or connectable to a tracker body. The second attachment portion includes a receptacle including an open ring. The receptacle is configured to receive and connect with either of the first or the second connection members through either one of opposing sides of the open ring.

In a ninth aspect, the surgical tracker assembly for tracking a bone is provided. The surgical tracker assembly includes a tracker body. The tracker body includes three arms extending in different directions from one another. Each arm supports a tracking element near a distal end of each arm. One of the three arms is a longest arm and includes a length that is greater than a length each of the other arms. The surgical tracker assembly also includes a first attachment portion coupled or connectable to the tracker body and defining a first keyed geometry. The surgical tracker assembly further includes a second attachment portion coupled or connectable to the bone and defining a second keyed geometry conforming to the first keyed geometry such that the second attachment portion receives the first attachment portion in a predefined orientation. The predefined orientation causes the longest arm of the tracker body to be oriented in a direction toward the bone.

In a tenth aspect, the surgical tracker assembly for tracking an object includes a tracker body and a mount assembly. The mount assembly includes a first attachment portion coupled or connectable to the tracker body. The first attachment portion includes a flexible connection member and a locking mechanism disposed adjacent to the flexible connection member. The mount assembly also includes a second attachment portion coupled or connectable to the object. The second attachment portion includes a receptacle including an open ring configured to receive the flexible connection member therethrough. The flexible connection member couples to the receptacle through a snap-fit, and the locking mechanism is configured to be rotated to bias the flexible connection member against the receptacle to lock the flexible connection member to the receptacle.

In an eleventh aspect, the surgical tracker assembly for tracking an object includes a tracker body and a mount assembly couplable to the tracker body. The tracker body includes a first connector. The mount assembly also includes a second connector engageable with the first connector of the tracker body. The surgical tracker assembly further includes a locking mechanism couplable to the tracker body and configured to secure the tracker body to the mount assembly. One of the first connector of the tracker body and the second connector of the mount assembly includes a receptacle extending between a first receptacle end and a second receptacle end. The first receptacle end defines a first opening and the second receptacle end defines a second opening. The other of the first connector of the tracker body and the second connector of the mount assembly includes a projection disposable in at least one of the first opening of the first receptacle end and the second opening of the second receptacle end to couple the tracker body to the mount assembly.

In a twelfth aspect, the surgical tracker assembly includes a tracker body and a mount assembly. The tracker body includes a first connector and the mount assembly includes a second connector releasably engageable with the first connector. One of the connectors is a rigid connector and the other of the connectors is a flexible connector configured to flex into engagement with the rigid connector. The rigid and flexible connectors are configured to couple to one another in at least two different poses.

In a thirteenth aspect, the surgical tracker assembly for tracking an object includes the tracker body including the first connector. The mount assembly is couplable to the tracker body. The mount assembly includes the second connector engageable with the first connector in at least a first position and a second position different from the first position. One of the first connector and the second connector is rigid, and the other of the first connector and the second connector is resilient.

In a fourteenth aspect, the surgical tracker assembly detectable by a localizer is provided. The surgical tracker assembly includes a support body and a tracker plate. The support body has an interface surface defining an open receptacle. The tracker plate includes a front side supporting a plurality of tracking elements detectable by the localizer. The tracker plate also includes a rear side facing away from the front side. The rear side includes a connector disposable in the open receptacle to couple the tracker plate to the interface surface of the support body.

In a fifteenth aspect, a coupler for a robotic surgical system is provided. The coupler includes a surgical tracker assembly detectable by a localizer. The surgical tracker assembly includes a support body having an interface surface defining an open receptacle, and a tracker plate including a front side supporting a plurality of tracking elements detectable by the localizer. The tracker plate also includes a rear side facing away from the front side, with the rear side including a connector disposable in the open receptacle to couple the tracker plate to the interface surface of the support body. The coupler also includes a coupler body configured to couple to the support body of the surgical tracker assembly. In one implementation, the support body is integral with the coupler body.

In a sixteenth aspect, a robotic surgical system for use with a localizer is provided. The robotic surgical system includes a coupler. The coupler includes a surgical tracker assembly detectable by a localizer. The surgical tracker assembly includes a support body having an interface surface defining an open receptacle, and a tracker plate including a front side supporting a plurality of tracking elements detectable by the localizer. The tracker plate also includes a rear side facing away from the front side, with the rear side including a connector disposable in the open receptacle to couple the tracker plate to the interface surface of the support body. The coupler also includes a coupler body configured to couple to the support body of the surgical tracker assembly. The robotic surgical system also includes a robotic manipulator including a plurality of links and a plurality of joints. One of the plurality of links is a distal link that includes a first mounting interface engageable with the first coupling interface of the coupler. The robotic surgical system further includes a tool including a second mounting interface engageable with the second coupling interface of the coupler.

In a seventeenth aspect, a tracker plate for a robotic surgical system for use with a localizer is provided. The tracker plate includes the front side supporting the plurality of tracking elements detectable by the localizer. The tracker plate also includes the rear side facing away from the front side. The rear side includes the first connector formed as the first resilient arm extending away from the rear side, and the first locating wall rigidly affixed to the rear side and disposed opposite the first connector. The rear side also includes the second connector formed as the second resilient arm extending away from the rear side, and the second locating wall rigidly affixed to the rear side and disposed opposite the second connector.

In some implementations, the multipositional receptacle defines a passageway between the first receptacle end and the second receptacle end. In some implementations, the projection is disposable through the passageway such that the projection is disposed through both the first opening of the first receptacle end and the second opening of the second receptacle end. In some implementations, the first receptacle end defining the first opening and the second receptacle end defining the second opening are symmetrical with respect to one another. In some implementations, the first receptacle end defining the first opening is symmetrical about a plane bisecting the first receptacle end and the first opening. In some implementations, the multipositional receptacle is further defined as a bipositional receptacle such that the tracker body is couplable to the mount assembly in only the first position and the second position.

In some implementations, the tracker body has a hub, a first arm extending away from the hub, a second arm extending away from the hub, and a third arm extending away from the hub, and the first connector extends away from the hub transverse to the first arm, the second arm, and the third arm. In some implementations, the first arm extends a first distance from the hub, the second arm extends a second distance from the hub, and the third arm extends a third distance from the hub, and the first distance is greater than either of the second distance and the third distance, and engagement of the first connector and the second connector orients the first arm to extend toward the object to be tracked. In some implementations, the tracker body has at least three arms extending away from the hub. In some implementations, the tracker body further has a fourth arm extending away from the hub, and the first connector extends away from the hub transverse to the fourth arm. In some implementations, the fourth arm extends a fourth distance from the hub, and the first distance is greater than the fourth distance. In some implementations, the tracker body further has a fifth arm extending away from the hub, and the first connector extends away from the hub transverse to the fifth arm. In some implementations, the fifth arm extends a fifth distance from the hub, and the first distance is greater than the fifth distance. In some implementations, the first connector is plastic, and the second connector is metallic.

In some implementations, the projection includes a first orientation feature and the multipositional receptacle includes a second orientation feature complementary to the first orientation feature of the projection, and alignment of the first orientation feature and the second orientation feature orients the tracker body relative to the mount assembly in a predefined orientation during engagement of the first connector and the second connector in the first position, and alignment of the first orientation feature and the second orientation feature orients the tracker body relative to the mount assembly in the predefined orientation during engagement of the first connector and the second connector in the second position. In some implementations, at least one of the first receptacle end and the second receptacle end has a keyed receptacle geometry that forms the second orientation feature, and the keyed receptacle geometry includes a curved portion proximal to the object to be tracked and a flat portion distal to the object to be tracked, and the projection has a keyed projection geometry that forms the first orientation feature, with the keyed projection geometry shaped to conform to the keyed receptacle geometry such that alignment of the keyed projection geometry and the keyed receptacle geometry orients the tracker body relative to the mount assembly in a predefined orientation, and the keyed projection geometry includes a flat portion distal to the object to be tracked and a curved portion proximal to the object to be tracked.

In some implementations, the projection includes a first resilient arm and a second resilient arm spaced from the first resilient arm. In some implementations, the multipositional receptacle presents an inner surface, and the first resilient arm and the second resilient arm are configured to engage the inner surface of the multipositional receptacle. In some implementations, the first resilient arm includes a first lip extending away from the second resilient arm and configured to abut the multipositional receptacle to retain the tracking body to the mount assembly, and the second resilient arm includes a second lip extending away from the first resilient arm and configured to abut the multipositional receptacle to retain the tracking body to the mount assembly.

In some implementations, the first lip tapers toward a distal end of the first resilient arm, the second lip tapers toward a distal end of the second resilient arm, the first resilient arm includes a non-tapered portion disposed between the first lip and the distal end of the first resilient arm, and the second resilient arm includes a non-tapered portion disposed between the second lip and the distal end of the second resilient arm. In some implementations, the multipositional receptacle includes a first narrowed portion alignable to abut the first lip of the first resilient arm and a second narrowed portion alignable to abut the second lip of the second resilient arm.

In some implementations, the first receptacle end includes a first receptacle end surface facing away from the second receptacle end, and the second receptacle end includes a second receptacle end surface facing away from the first receptacle end, and the first narrowed portion presents a first narrowed surface indented relative to one of the first receptacle end surface and the second receptacle end surface, and the second narrowed portion presents a second narrowed surface indented relative to the one of the first receptacle end surface and the second receptacle end surface. In some implementations, the first narrowed portion presents a third narrowed surface indented relative to the other of the first receptacle end surface and the second receptacle end surface, and the second narrowed portion presents a fourth narrowed surface indented relative to the other of the first receptacle end surface and the second receptacle end surface.

In some implementations, the tracker body includes a first stabilizing projection extending toward the multipositional receptacle and shaped to correspond to the multipositional receptacle, and the tracker body includes a second stabilizing projection extending toward the multipositional receptacle and shaped to correspond to the multipositional receptacle. In some implementations, the first stabilizing projection and the second stabilizing projection are circumferentially offset relative to the first resilient arm and the second resilient arm. In some implementations, the first stabilizing projection and the second stabilizing projection taper toward the multipositional receptacle.

In some implementations, the multipositional receptacle presents an inner surface, and the first narrowed surface tapers toward the inner surface of the multipositional receptacle, and the second narrowed surface tapers toward the inner surface of the multipositional receptacle. In some implementations, the first lip includes a first abutment surface shaped to correspond to the first narrowed surface, and the second lip includes a second abutment surface shaped to correspond to the second narrowed surface. In some implementations, the first resilient arm is moveable toward the second resilient arm and the second resilient arm is moveable toward the first resilient arm to permit the projection to be disposed in at least one of the first opening and the second opening of the multipositional receptacle. In some implementations, the first resilient arm and the second resilient arm are configured to engage the multipositional receptacle through a snap-fit.

In some implementations, the surgical tracker assembly further includes a locking mechanism couplable to the tracker body and configured to secure the tracker body to the mount assembly. In some implementations, the locking mechanism includes a stop disposed between the first resilient arm and the second resilient arm, and the stop is moveable between a first stop position where the first resilient arm and the second resilient arm are permitted to move toward one another, and a second stop position where the first resilient arm and the second resilient arm are limited from moving toward one another. In some implementations, the stop includes a weakened region, and the stop is breakable at the weakened region. In some implementations, the weakened region has a reduced diameter relative to adjacent portions of the stop. In some implementations, the stop is configured to break at the weakened region in response to rotation of the stop from the first stop position to the second stop position, and the stop is configured to remain disposed between the first resilient arm and the second resilient arm upon breaking. In some implementations, the first connector includes the projection and the second connector includes the multipositional receptacle.

In some implementations, the support body includes a support plate extending approximately parallel to the tracker plate, and the support plate has the interface surface defining the open receptacle. In some implementations, the connector is further defined as a resilient arm. In some implementations, the connector is engageable with the support body through a snap-fit. In some implementations, the rear side of the tracker plate further includes a locating wall disposable in the open receptacle, with the locating wall being rigidly affixed relative to the tracker plate. In some implementations, the connector and the locating wall are disposed opposite one another when disposed in the open receptacle. In some implementations, the connector is configured to bias the locating wall against the support body in a first direction.

In some implementations, the connector is further defined as a first connector, and the rear side of the tracker plate further includes a second connector disposable in the open receptacle to couple the tracker plate to the interface surface of the support body. In some implementations, the second connector is arranged circumferentially spaced from the first connector relative to the open receptacle. In some implementations, the rear side of the tracker plate further includes a second locating wall disposable in the open receptacle, with the second locating wall being rigidly affixed relative to the tracker plate. In some implementations, the second connector and the second locating wall are disposed opposite one another when disposed in the open receptacle.

In some implementations, the rear side of the tracker plate further includes a locating wall disposable in the open receptacle, with the locating wall being rigidly affixed relative to the tracker plate, and the connector is configured to bias the locating wall against the support body in a first direction, and the connector is further defined as a first connector, and the rear side of the tracker plate further includes a second connector disposable in the open receptacle to couple the tracker plate to the interface surface of the support body, and the rear side of the tracker plate further includes a second locating wall disposable in the open receptacle, with the second locating wall being rigidly affixed relative to the tracker plate, and the second connector is configured to bias the second locating wall against the support body in a second direction circumferentially spaced from the first direction.

In some implementations, the interface surface of the support body defines a second open receptacle, and the rear side of the tracker plate includes a third connector disposable in the second open receptacle to couple the tracker plate to the interface surface of the support body. In some implementations, the rear side of the tracker plate further includes a first locating wall disposable in the open receptacle, with the first locating wall being rigidly affixed relative to the tracker plate, and the rear side of the tracker plate further includes a third locating wall disposable in the second open receptacle, with the third locating wall being rigidly affixed relative to the tracker plate. In some implementations, the third connector and the third locating wall are disposed opposite one another when disposed in the second open receptacle. In some implementations, the connector is configured to bias the first locating wall against the support body in a first direction, and the third connector is configured to bias the third locating wall against the support body in the first direction. In some implementations, the first locating wall and the third locating wall together approximate a line of contact between the tracker plate and the support body.

In some implementations, the rear side of the tracker plate includes a fourth connector disposable in the second open receptacle to couple the tracker plate to the interface surface of the support body. In some implementations, the rear side of the tracker plate is free of a fourth locating wall disposable in the second open receptacle opposite the fourth connector.

In some implementations, the rear side of the tracker plate further includes a locating wall disposable in the open receptacle, with the locating wall being rigidly affixed relative to the tracker plate, and the connector is configured to bias the locating wall against the support body in a first direction, and the connector is further defined as a first connector, and the rear side of the tracker plate further includes a second connector disposable in the open receptacle to couple the tracker plate to the interface surface of the support body, and the rear side of the tracker plate further includes a second locating wall disposable in the open receptacle, with the second locating wall being rigidly affixed relative to the tracker plate, and the second connector is configured to bias the second locating wall against the support body in a second direction circumferentially spaced from the first direction, and the interface surface of the support body defines a second open receptacle, and the rear side of the tracker plate includes a third connector disposable in the second open receptacle to couple the tracker plate to the interface surface of the support body, and the rear side of the tracker plate includes a fourth connector disposable in the second open receptacle to couple the tracker plate to the interface surface of the support body, and the fourth connector is configured to bias the tracker plate in the second direction.

In some implementations, the tracker plate includes a first orientation feature and the support body includes a second orientation feature complementary to the first orientation feature of the tracker plate, and alignment of the first orientation feature and the second orientation feature orients the tracker plate relative to the support body in a predefined orientation. In some implementations, the tracker plate includes a keyed projection that forms the first orientation feature, and the support body defines a keyed receptacle that forms the second orientation feature, and the keyed projection of the tracker plate is disposable at least partially in the keyed receptacle of the support body to orient the tracker plate relative to the support body.

In some implementations, the support body is metallic and the tracker plate is plastic. In some implementations, the tracker plate is detachable from the support body. In some implementations, the tracker plate includes a backing plate having the rear side, and includes a face plate having the front side, and the face plate and the backing plate are affixed together. In some implementations, the backing plate defines a connector aperture, and the connector includes a parallel portion extending parallel to the tracker plate and into the connector aperture, and the connector includes a perpendicular portion extending from the parallel portion away from the backing plate. In some implementations, the support body includes a first support arm extending away from the support plate and configured to couple to a coupler, and the support body includes a second support arm extending away from the support plate and configured to couple to the coupler.

Any of the above aspects can be combined in full or in part. Any features of the above aspects can be combined in full or in part. Any of the above implementations can be combined, in full or in part, with any other aspect. Any of the above implementations can be combined with any other implementation whether for the same aspect or different aspect.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a robotic surgical system, according to one implementation;

FIG. 2 is a block diagram of a control system for controlling the robotic surgical system, according to one implementation;

FIG. 3A is a perspective view of a surgical tracker assembly arranged to engage in a first position, according to one implementation;

FIG. 3B is a perspective view of the surgical tracker assembly arranged to engaged in a second position, according to one implementation;

FIG. 4A is a perspective view of the surgical tracker assembly engaged in the first position, according to one implementation;

FIG. 4B is a perspective view of the surgical tracker assembly engaged in the second position, according to one implementation;

FIG. 5A is a cross-sectional view of the surgical tracker assembly of FIGS. 3A-4B, according to one implementation;

FIG. 5B is a cross-sectional view of the surgical tracker assembly of FIGS. 3A-4B, taken approximately 90 degrees relative to the cross-sectional view of FIG. 5A, according to one implementation;

FIG. 6A is a frontal view of the surgical tracker assembly, with the surgical tracker assembly including a locking mechanism having a stop in a first stop position, according to one implementation;

FIG. 6B is a frontal view of the surgical tracker assembly, with the surgical tracker assembly including the locking mechanism having the stop in a second stop position, according to one implementation;

FIG. 7 is a cross-sectional view of the surgical tracker assembly of FIGS. 6A and 6B, according to one implementation;

FIG. 8 is a perspective view of a surgical tracker assembly, according to another implementation;

FIG. 9 is a bottom view of the surgical tracker assembly of FIG. 8, according to one implementation; and

FIG. 10 is a back view of the surgical tracker assembly of FIGS. 8 and 9, according to one implementation.

### DETAILED DESCRIPTION

### I. Example Surgical System

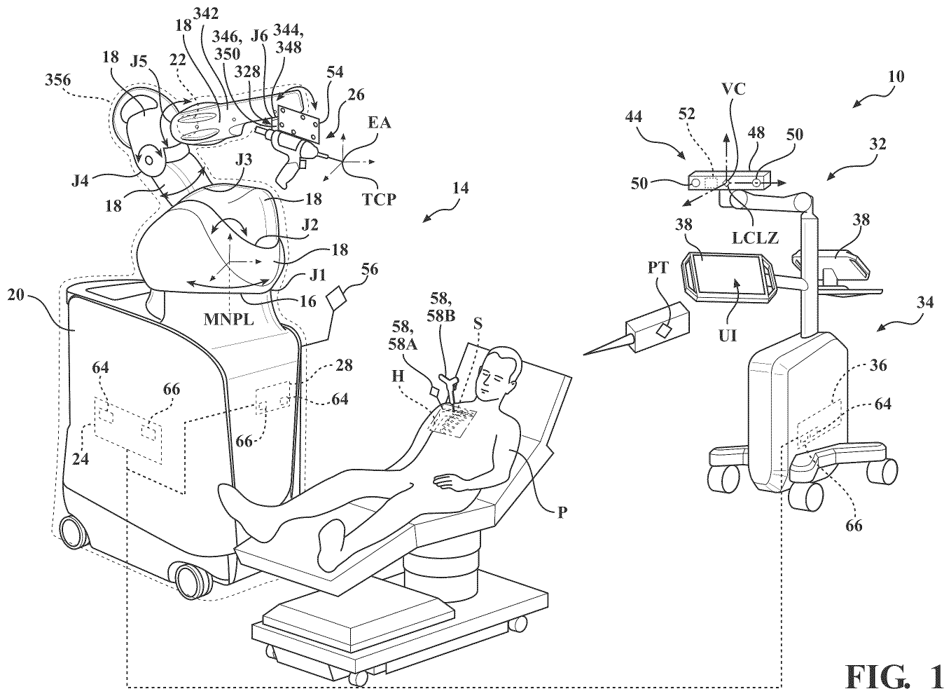

Referring to FIG. 1, a robotic surgical system 10 is illustrated. The robotic surgical system 10 is useful for treating a surgical site or anatomical volume of a patient P, such as treating bone or soft tissue. In FIG. 1, the patient P is undergoing a surgical procedure. The surgical procedure may involve tissue removal or other forms of treatment. Treatment may include cutting, coagulating, lesioning the tissue, other in-situ tissue treatments, or the like. In some examples, the surgical procedure involves shoulder replacement surgery, partial or total knee or hip replacement surgery, spine surgery, or ankle surgery. In some examples, the system 10 is designed to cut away material to be replaced by surgical implants, such as shoulder implants, partial or total knee implants, hip implants, spine implants, or ankle implants. In FIG. 1, the system 10 is shown being employed to prepare the humerus H and/or a glenoid cavity of a scapula S to receive shoulder implants. Examples of shoulder implants, and methods of implanting them, are shown in U.S. Patent Publication No. 2022/0039898, filed on Aug. 4, 2021, entitled, “Robotic Surgical System Including a Coupler for Connecting a Tool to a Manipulator and Methods of Using the Coupler,” U.S. Pat. No. 11,432,945, filed on Nov. 6, 2018, entitled, “Robotic System For Shoulder Arthroplasty Using Stemless Implant Components,” and U.S. Pat. No. 11,173,048, filed on Nov. 6, 2018, entitled, “Robotic System For Shoulder Arthroplasty Using Stemless Implant Components,” the disclosures of both of which are hereby incorporated herein by reference. The system 10 and techniques disclosed herein may be used to perform other procedures, surgical or non-surgical, or may be used in industrial applications or other applications where robotic systems are utilized.

The system 10 may include a robotic manipulator 14. The manipulator 14 has a base 16 and plurality of links 18. A manipulator cart 20 can support the manipulator 14 such that the manipulator 14 is fixed to the manipulator cart 20. In other examples, the manipulator 14 can be mounted to a surgical patient table. The links 18 collectively form one or more arms or linkages of the manipulator 14 with adjacent links being connected by joints. The manipulator 14 may have a serial, robotic arm configuration (as shown in FIG. 1), a parallel, robotic arm configuration, or any other suitable manipulator configuration. In other examples, more than one manipulator 14 may be utilized in a multiple arm configuration.

In the example shown in FIG. 1, the manipulator 14 comprises a plurality of joints J1-J6 and a plurality of joint encoders 22 located at the joints J1-J6 for determining position data (e.g., rotation angles) of the joints J1-J6. For simplicity, only one joint encoder 22 is illustrated in FIG. 1, although other joint encoders 22 may be similarly illustrated. The manipulator 14 according to one example has six joints J1-J6 implementing at least six-degrees of freedom (DOF) for the manipulator 14. However, the manipulator 14 may have any number of degrees of freedom and may have any suitable number of joints J and may have redundant joints.

The manipulator 14 need not require joint encoders 22 but may alternatively, or additionally, utilize motor encoders present on motors at each joint J. Also, the manipulator 14 need not require rotary joints, but may alternatively, or additionally, utilize one or more prismatic joints. Any suitable combination of joint types are contemplated.

The base 16 of the manipulator 14 is generally a portion of the manipulator 14 that provides a fixed reference coordinate system for other components of the manipulator 14 or the system 10 in general. Generally, the origin of a manipulator coordinate system MNPL is defined at the fixed reference of the base 16. The base 16 may be defined with respect to any suitable portion of the manipulator 14, such as one or more of the links 18. Alternatively, or additionally, the base 16 may be defined with respect to the manipulator cart 20, such as where the manipulator 14 is physically attached to the cart 20. In one example, the base 16 is defined at an intersection of the axes of joints J1 and J2. Thus, although joints J1 and J2 are moving components in reality, the intersection of the axes of joints J1 and J2 is nevertheless a virtual fixed reference pose, which provides both a fixed position and orientation reference and which does not move relative to the manipulator 14 and/or manipulator cart 20. In other examples, the manipulator 14 can be a hand-held manipulator where the base 16 is a base portion of a tool (e.g., a portion held free-hand by the user) and the tool tip is movable relative to the base portion. The base portion has a reference coordinate system that is tracked and the tool tip has a tool tip coordinate system that is tracked relative to the reference coordinate system.

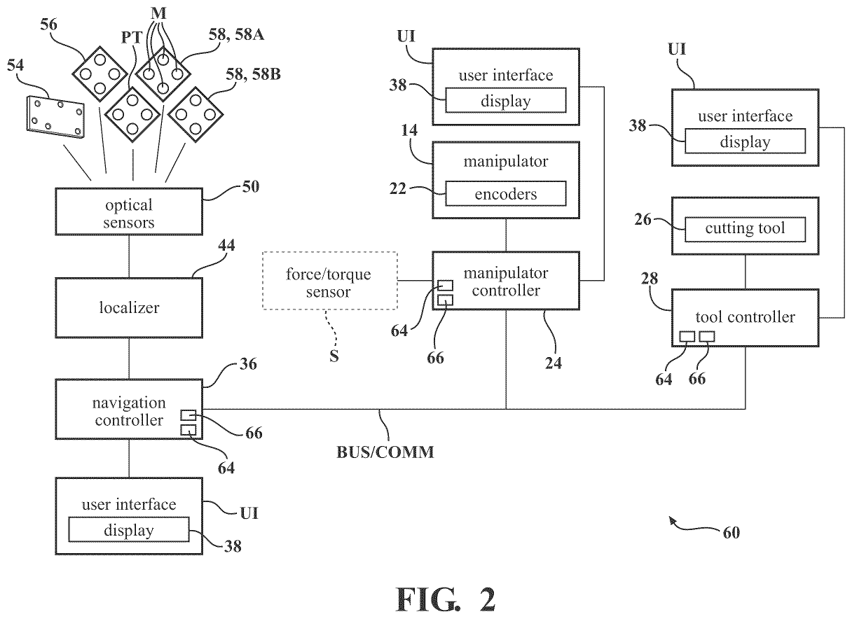

The manipulator 14 and/or manipulator cart 20 house a manipulator controller 24, or other type of control unit. The manipulator controller 24 may comprise one or more computers, or any other suitable form of controller that directs the motion of the manipulator 14. The manipulator controller 24 may have a central processing unit (CPU) and/or other processors, memory (not shown), and storage (not shown). The manipulator controller 24 is loaded with software as described below. The processors could include one or more processors to control operation of the manipulator 14. The processors can be any type of microprocessor, multi-processor, and/or multi-core processing system. The manipulator controller 24 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any embodiment to a single processor. The manipulator 14 may also comprise a user interface UI (see FIG. 2) with one or more displays and/or input devices (e.g., push buttons, keyboard, mouse, microphone (voice-activation), gesture control devices, touchscreens, etc.).

A surgical tool 26 is coupled, or can be releasably attached, to the manipulator 14 and is movable relative to the base 16 to interact with the anatomy. The tool 26 is or forms part of an end effector supported by the manipulator 14 in certain embodiments. The tool 26 may be grasped by the user. One possible arrangement of the manipulator 14 and the tool 26 is described in U.S. Pat. No. 9,119,655, entitled, “Surgical Manipulator Capable Of Controlling A Surgical Tool In Multiple Modes,” filed on Aug. 2, 2013, the disclosure of which is hereby incorporated herein by reference. The manipulator 14 and the tool 26 may be arranged in alternative configurations. The tool 26 can be like that shown in U.S. Pat. No. 9,566,121, filed on Mar. 15, 2014, entitled, “End Effector Of A Surgical Robotic Manipulator,” hereby incorporated herein by reference.

The tool 26 includes an energy applicator EA designed to interact with the patient P. In some instances, the tool 26 can contact and remove the tissue of the patient P at the surgical site. In one example, the energy applicator EA is a burr. The burr may be substantially spherical and comprise a spherical center, radius (r) and diameter. Alternatively, the energy applicator EA may be a drill bit, a saw blade, an ultrasonic vibrating tip, or the like. In some versions, the tool 26 includes non-motorized accessories such as a probe, a retractor, a cutting guide, or the like. The tool 26 and/or energy applicator EA/accessory may comprise any geometric feature, e.g., perimeter, circumference, radius, diameter, width, length, volume, area, surface/plane, range of motion envelope (along any one or more axes), etc. The geometric feature may be considered to determine how to locate the tool 26 relative to the tissue at the surgical site to perform the desired treatment. In some of the embodiments described herein, a spherical burr having a tool center point (TCP) will be described for convenience and case of illustration but is not intended to limit the tool 26 to any particular form. The tool 26 may include a tool driver 26 that houses any driving motor for the energy applicator EA, e.g., to drive saw blade oscillation, burr rotation, drill rotation, etc. The tool 26 may also include a tool holder that releasably connects to the tool driver to interchange different energy applicators EA. The tool holder releasably holds the energy applicator EA. The tool holder can be releasably connected to the tool driver using any suitable type of connection, e.g., snap-fit connection, bayonet-type connection, fasteners, collet connections, or the like.

The tool 26 may comprise a tool controller 28 to control operation of the tool 26, such as to control power to the tool (e.g., to a rotary, driving motor of the tool 26), control movement of the tool 26, control irrigation/aspiration of the tool 26, and/or the like. The tool controller 28 may be in communication with the manipulator controller 24 or other components. The tool 26 may also comprise a user interface UI with one or more displays and/or input devices (e.g., push buttons, keyboard, mouse, microphone (voice-activation), gesture control devices, touchscreens, etc.). The manipulator controller 24 controls a state (e.g., position and/or orientation) of the tool 26 (e.g., the TCP) with respect to a coordinate system, such as the manipulator coordinate system MNPL. The manipulator controller 24 can control (linear or angular) velocity, acceleration, or other derivatives of motion of the tool 26.

The tool center point (TCP), in one example, is a predetermined reference point or coordinate system defined at the energy applicator EA. The TCP has a known, or able to be calculated (i.e., not necessarily static), pose relative to other coordinate systems. The geometry of the energy applicator EA is known in or defined relative to a TCP coordinate system. The TCP may be located at the spherical center of the burr of the tool 26 such that only one point is tracked. The TCP may be defined in various ways depending on the configuration of the energy applicator EA. The manipulator 14 could employ the joint/motor encoders, or any other non-encoder position sensing method, to enable a pose of the TCP to be determined. The manipulator 14 may use joint measurements to determine TCP pose and/or could employ techniques to measure TCP pose directly. The control of the tool 26 is not limited to a center point. For example, any suitable primitives, meshes, etc., can be used to represent the tool 26.

The system 10 further includes a navigation system 32. One example of the navigation system 32 is described in U.S. Pat. No. 9,008,757, filed on Sep. 24, 2013, entitled, “Navigation System Including Optical And Non-Optical Sensors,” hereby incorporated herein by reference. The navigation system 32 tracks movement of various objects. Such objects include, for example, the manipulator 14, the tool 26 and the anatomy, e.g., the humerus H and scapula S. The navigation system 32 tracks these objects to gather state information of each object with respect to a (navigation) localizer coordinate system LCLZ. Coordinates in the localizer coordinate system LCLZ may be transformed to the manipulator coordinate system MNPL, and/or vice-versa, using transformations.

The navigation system 32 includes a cart assembly 34 that houses a navigation controller 36, and/or other types of control units. A navigation user interface UI is in operative communication with the navigation controller 36. The navigation user interface includes one or more displays 38. The navigation system 32 is capable of displaying a graphical representation of the relative states of the tracked objects to the user using the one or more displays 38. The navigation user interface UI further comprises one or more input devices to input information into the navigation controller 36 or otherwise to select/control certain aspects of the navigation controller 36. Such input devices include interactive touchscreen displays. However, the input devices may include any one or more of push buttons, a keyboard, a mouse, a microphone (voice-activation), gesture control devices, and the like.

The navigation system 32 also includes a navigation localizer 44 coupled to the navigation controller 36. In one example, the localizer 44 is an optical localizer and includes a camera unit 46. The camera unit 46 has an outer casing 48 that houses one or more optical sensors 50. The localizer 44 may include its own localizer controller 52 and may further include a video camera VC.

The navigation system 32 includes one or more surgical tracker assemblies. In one example, the surgical tracker assemblies include a pointer tracker PT, a surgical tracker assembly 54 for the tool 26, a surgical tracker assembly 56 for the manipulator 14, one or more surgical tracker assemblies 58 for the patient, including surgical tracker 58A for the patient and surgical tracker assembly 58B for the patient. In the illustrated example of FIG. 1, the surgical tracker assembly 54 is fixed with respect to the tool 26, the surgical tracker assembly 58A is firmly affixed to the humerus H of the patient P, and the surgical tracker assembly 58B is firmly affixed to the scapula S of the patient P. In this example, the surgical tracker assemblies 58A, 58B for the patient are firmly affixed to sections of bone. The pointer tracker PT is firmly affixed to a pointer used for registering the anatomy to the localizer coordinate system LCLZ. The surgical tracker assembly 56 may be affixed to any suitable component of the manipulator 14, in addition to, or other than the tool 26, such as the base 16, the cart 20, or any one or more links 18 of the manipulator 14. The surgical tracker assemblies 54, 56, 58A, 58B, PT may be fixed to their respective components in any suitable manner. For example, the trackers may be rigidly fixed, flexibly connected (optical fiber), or not physically connected at all (ultrasound), as long as there is a suitable (supplemental) way to determine the relationship (measurement) of that respective tracker to the object that it is associated with. In some cases, only one of the surgical tracker assemblies 54, 56 for the tool 26 and the manipulator 14 are used, or both the surgical tracker assemblies 54, 56 for the tool 26 and the manipulator 14 may be used.

In the illustrated embodiment, the surgical tracker assemblies 54, 56, 58A, 58B, PT are passive trackers. Accordingly, each surgical tracker assembly 54, 56, 58A, 58B, PT has at least three passive tracking elements, fiducials, or markers M, such as reflectors, for reflecting light from the localizer 44 back to the optical sensors 50. In other embodiments, the surgical tracker assemblies 54, 56, 58A, 58B, PT are active trackers and may have light emitting diodes or LEDs transmitting light, such as infrared light to the optical sensors 50. Based on the received optical signals, navigation controller 36 generates data indicating the relative positions and orientations of the surgical tracker assemblies 54, 56, 58A, 58B, PT relative to the localizer 44 using conventional triangulation techniques. In some cases, more or fewer markers may be employed. For instance, in cases in which the object being tracked is rotatable about a line, two markers can be used to determine an orientation of the line by measuring positions of the markers at various locations about the line. It should be appreciated that the localizer 44 and surgical tracker assemblies 54, 56, 58A, 58B, PT, although described above as utilizing optical tracking techniques, could alternatively, or additionally, utilize other tracking modalities to track the objects, such as electromagnetic tracking, radio frequency tracking, inertial tracking, ultrasound-based tracking, fiber-optic tracking, machine-vision tracking, combinations thereof, and the like.

The localizer 44 tracks the surgical tracker assemblies 54, 56, 58A, 58B, PT to determine a state of each of the surgical tracker assemblies 54, 56, 58A, 58B, PT, which correspond respectively to the state of the object respectively attached thereto. The localizer 44 provides the state of the surgical tracker assemblies 54, 56, 58A, 58B, PT to the navigation controller 36. In one example, the navigation controller 36 determines and communicates the state of the surgical tracker assemblies 54, 56, 58A, 58B, PT to the manipulator controller 24. As used herein, the state of an object includes, but is not limited to, data that defines the position and/or orientation of the tracked object or equivalents/derivatives of the position and/or orientation. For example, the state may be a pose of the object, and may include linear velocity data, and/or angular velocity data, and the like.

The navigation controller 36 may comprise one or more computers, or any other suitable form of controller. Navigation controller 36 has a central processing unit (CPU) and/or other processors, memory (not shown), and storage (not shown). The processors can be any type of processor, microprocessor or multi-processor system. The navigation controller 36 is loaded with software. The software, for example, converts the signals received from the localizer 44 into data representative of the position and orientation of the objects being tracked. The navigation controller 36 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any embodiment to a single processor.

In operation, for certain surgical tasks, the user manually manipulates (e.g., moves or causes the movement of) the tool 26 to perform the surgical procedure on the patient, such as drilling, cutting, sawing, reaming, implant installation, and the like. As the user manipulates the tool 26, the navigation system 32 tracks the location of the tool 26 and/or the manipulator 14 and provides haptic feedback (e.g., force feedback) to the user to limit the user's ability to move (or cause movement of) the tool 26 beyond one or more predefined virtual boundaries that are registered (or mapped) to the patient's anatomy, which results in highly accurate and repeatable drilling, cutting, sawing, reaming, and/or implant placement.

In some embodiments, the manipulator 14 operates in a passive manner and provides haptic feedback when the surgeon attempts to move the tool 26 beyond the virtual boundary. The haptic feedback (e.g., a form of stereotactic feedback) is generated by one or more actuators (e.g., joint motors) of the manipulator 14 and transmitted to the user via a flexible transmission, such as a cable drive transmission. When the manipulator 14 is not providing haptic feedback, the manipulator 14 is freely moveable by the user. In some embodiments, like that shown in U.S. Pat. No. 9,566,122, incorporated herein by reference, the manipulator 14 is manipulated by the user in a similar manner, but the manipulator 14 operates in an active manner. For instance, the user applies force to the tool 26, which is measured by a force/torque sensor S (see FIG. 2), and the manipulator 14 emulates the user's desired movement based on measurements from the force/torque sensor S. For other surgical tasks, the manipulator 14 may operate autonomously.

Referring to FIG. 2, the system 10 includes a control system 60 that comprises, among other components, the manipulator controller 24, the navigation controller 36, and the tool controller 28. The control system 60 further includes one or more software programs and software modules. The software modules may be part of the program or programs that operate on the manipulator controller 24, navigation controller 36, tool controller 28, or any combination thereof, to process data to assist with control of the system 10. The software programs and/or modules include computer readable instructions stored in non-transitory memory 64 on the manipulator controller 24, navigation controller 36, tool controller 28, or a combination thereof, to be executed by one or more processors 66 of the controllers 24, 28, 36. The memory 64 may be any suitable configuration of memory, such as RAM, non-volatile memory, etc., and may be implemented locally or from a remote database. Additionally, software modules for prompting and/or communicating with the user may form part of the program or programs and may include instructions stored in memory 64 on the manipulator controller 24, navigation controller 36, tool controller 28, or any combination thereof. The user may interact with any of the input devices of the navigation user interface UI or other user interface UI to communicate with the software modules. The user interface software may run on a separate device from the manipulator controller 24, navigation controller 36, and/or tool controller 28.

The control system 60 may comprise any suitable configuration of input, output, and processing devices suitable for carrying out the functions and methods described herein. The control system 60 may comprise the manipulator controller 24, the navigation controller 36, or the tool controller 28, or any combination thereof, or may comprise only one of these controllers. These controllers may communicate wirelessly, via a bus as shown in FIG. 2, or otherwise. The control system 60 may also be referred to as a controller. The control system 60 may comprise one or more microcontrollers, field programmable gate arrays, systems on a chip, discrete circuitry, sensors, displays, user interfaces, indicators, and/or other suitable hardware, software, or firmware that is capable of carrying out the functions described herein.

The manipulator controller 24 and/or the navigation controller 36 track the state of the tool 26 relative to the anatomy and the virtual boundaries. In one example, the state of the TCP is measured relative to the virtual boundaries for purposes of determining haptic forces to be applied to a virtual rigid body model via a virtual simulation so that the tool 26 remains in a desired positional relationship to the virtual boundaries (e.g., not moved beyond them, kept within them, etc.). The results of the virtual simulation are commanded to the manipulator 14.

In some implementations, using the navigation system 32, the pose of the tool 26 can be determined by tracking the location of the base 16 and the associated manipulator coordinate system MNPL via the surgical tracker assembly 56 for the manipulator 14 and calculating the pose of the tool 26 based on joint encoder data from the joint encoders 22 (and/or motor encoders) at the joints J1-J6 (using kinematic data) and based on a known geometric relationship between the tool 26 and the manipulator 14. Ultimately, the localizer 44 and the surgical tracker assemblies 54, 56, 58A, 58B, PT enable the determination of the pose of the tool 26 and the patient's anatomy so the navigation system 32 knows the relative relationship between the tool 26 and the patient's anatomy. However, in some cases, the surgical tracker assembly 56 for the manipulator 14 may be out of view of the localizer 44, or the surgical tracker assembly 56 for the manipulator 14 may not be used. Line-of-sight between one or more of the sensors 50 and the surgical tracker assembly 56 for the manipulator 14 may be obstructed such that movement of the tool 26 cannot be reliably tracked solely using the surgical tracker assembly 56 for the manipulator 14 and encoder data. In this case, the surgical tracker assembly 54 for the tool 26 can be employed to track movement of the tool 26, i.e., the surgical tracker assembly 54 for the tool 26 is detected by the localizer 44 to determine a pose of the tool 26 (e.g., of the TCP coordinate system of the tool 26).

### II. Surgical Tracker Assembly Configurations

Described in this section are example surgical tracker assemblies that provide a technical solution for many of the problems known with conventional trackers. For example, the surgical tracker assemblies described herein have minimized and optimized footprints that minimize interference with the surgical site and unwanted collisions. The surgical tracker assemblies described herein are lightweight, which enables more stable installation to the object being tracked and potentially less fatigue to a surgeon who may need to support the tracker assemblies while using the tracked object. The surgical tracker assemblies accomplish the minimized footprint and lightweight design while simultaneously optimizing the number of tracking markers supported by the tracker assembly to increase tracking accuracy by the localizer. Furthermore, the tracker assemblies described herein are low-profile and have virtually zero tolerance stack. Furthermore, the surgical tracker assemblies described herein do not require hermetic sealing to protect internal components of the tracker assembly, which reduces complications and costs. For many of the reasons above, the surgical tracker assemblies described herein are suitable to be disposable or single use components that do not require exposure to the sterilization process. The surgical tracker assemblies described herein reduce the number of components and complexity associated with creating a rigid connection the object. In some examples, the tracker assemblies include integrated features to enable a “snap fit” directly to the supporting components. In some instances, the tracker assemblies described herein enable repeatable connection to the support component, and in multiple directions, without the need to perform cumbersome adjustments, such as precisely aligning indexing features, or precisely positioning the tracker and holding the tracker stationary while simultaneously securing fasteners or clamps. Other advantages of the tracker assemblies described herein will be fully understood from the detailed description and figures.

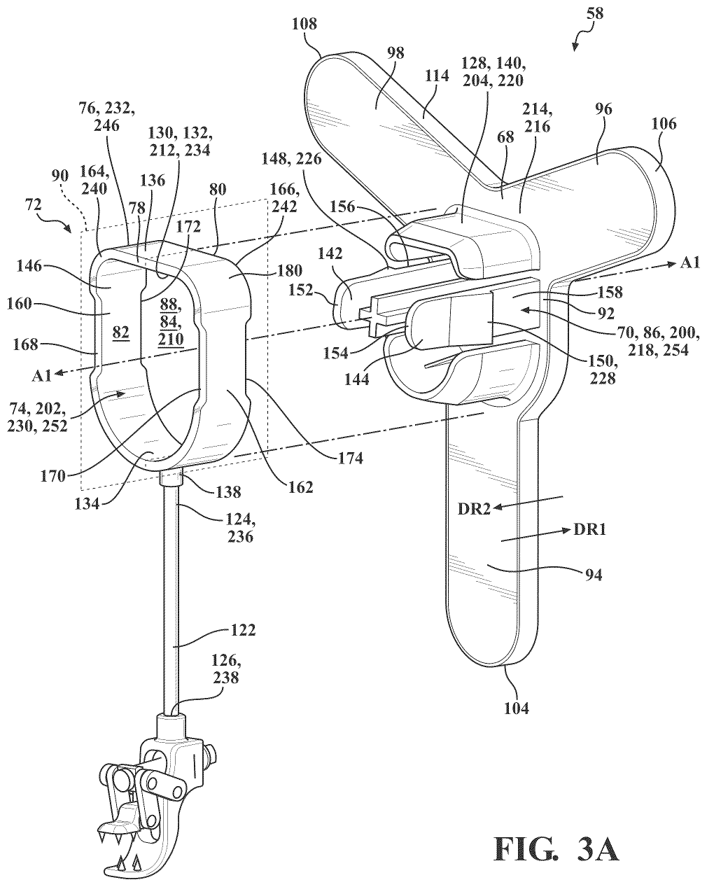

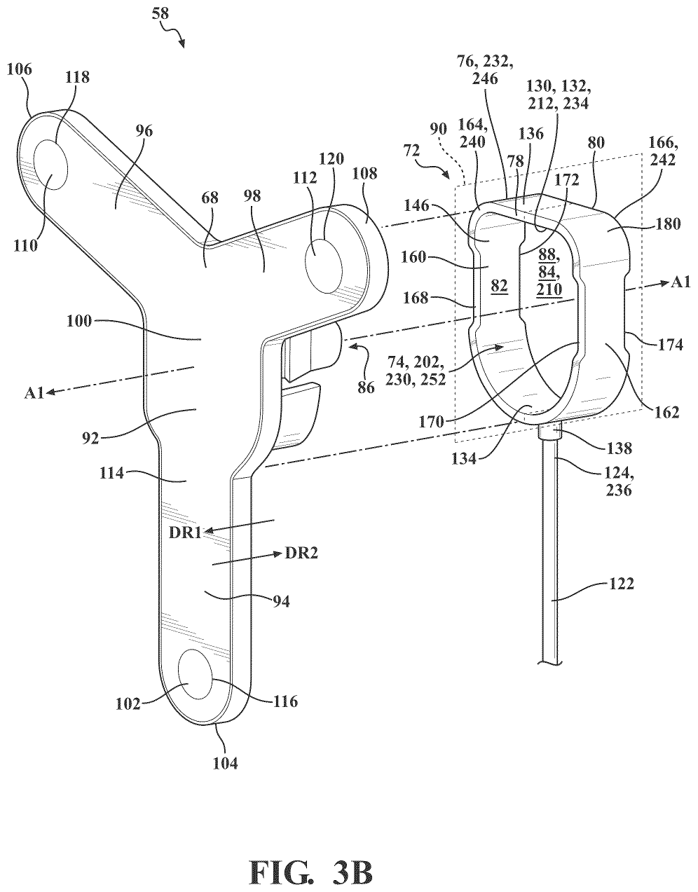

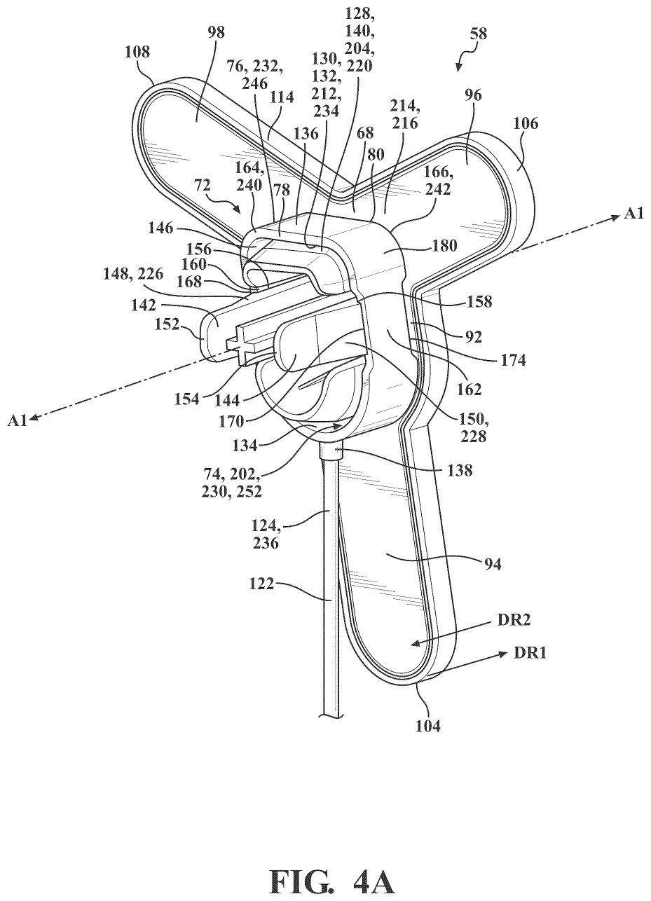

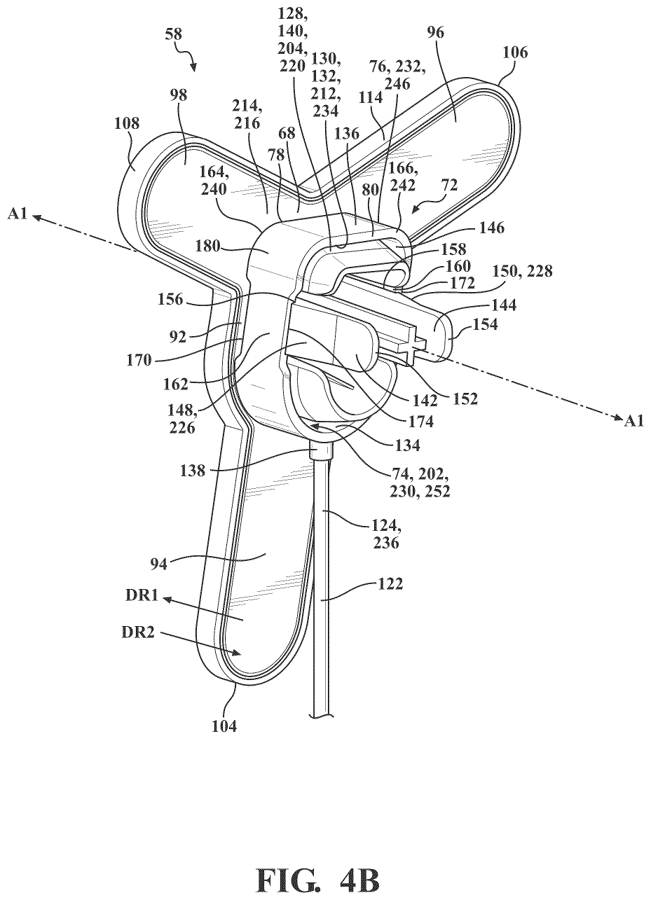

With reference to FIG. 3A-6, wherein like numerals indicate like parts throughout the several views, a surgical tracker assembly 58 for tracking an object includes a tracker body 68 including a first connector 70 and a mount assembly 72 couplable to the tracker body 68. The mount assembly 72 includes a second connector 74 engageable with the first connector 70 in at least a first position and a second position different from the first position. An exemplary depiction of the second connector 74 preparing to engage the first connector 70 is shown in FIG. 3A, and an exemplary depiction of the second connector 74 preparing to engage the first connector 70 is shown in FIG. 3B. An exemplary depiction of the second connector 74 engaged with the first connector 70 in the first position is shown in FIG. 4A, and an exemplary depiction of the second connector 74 engaged with the first connector 70 in the second position is shown in FIG. 4B.

One of the first connector 70 and the second connector 74 includes a multipositional receptacle 76. In other words, the first connector 70 may include the multipositional receptacle 76 or the second connector 74 may include the multipositional receptacle 76. The multipositional receptacle 76 extends between a first receptacle end 78 and a second receptacle end 80. The first receptacle end 78 defines a first opening 82 and the second receptacle end 80 defines a second opening 84. The other of the first connector 70 and the second connector 74 includes a projection 86. In other words, in one implementation the first connector 70 includes the multipositional receptacle 76 and the second connector 74 includes the projection 86, and in another implementation the first connector 70 includes the projection 86 and the second connector 74 includes the multipositional receptacle 76.

The projection 86 is disposable in the first opening 82 of the first receptacle end 78 to couple the tracker body 68 to the mount assembly 72 in the first position, and the projection 86 is disposable in the second opening 84 of the second receptacle end 80 to couple the tracker body 68 to the mount assembly 72 in the second position. The projection 86 being disposable in either the first opening 82 or the second opening 84 to couple the tracker body 68 to the mount assembly 72 in either the first position or the second position, respectively, advantageously permits the surgical tracker assembly 58 to be used for surgical procedures which on bodily anatomy which has a symmetrical pair in the human body. As a non-limiting example, the projection 86 being disposable in either the first opening 82 or the second opening 84 to couple the tracker body 68 to the mount assembly 72 in either the first position or the second position, respectively, advantageously permits the surgical tracker assembly 58 to be used for shoulder surgery, and more specifically, for surgery on the right shoulder of the patient or the left shoulder of the patient.

In one implementation, the multipositional receptacle is solid between the first opening 82 of the first receptacle end 78 and the second opening 84 of the second receptacle end 80. However, in another implementation, the multipositional receptacle 76 may define a passageway 88 between the first receptacle end 78 and the second receptacle end 80. As such, the passageway 88 may extend completely through the multipositional receptacle 76, and the multipositional receptacle 76 may be defined as a through-ring. The projection 86 may be disposable through the passageway 88 such that the projection 86 is disposed through both the first opening 82 of the first receptacle end 78 and the second opening 84 of the second receptacle end 80. In other words, in the first position, the projection 86 may extend into the first opening 82, through the passageway 88, and out of the second opening 84. Moreover, in the second position, the projection 86 may extend into the second opening 84, through the passageway 88, and out of the first opening 82.

The first receptacle end 78 defining the first opening 82 and the second receptacle end 80 defining the second opening 84 may be symmetrical with respect to one another. Additionally, the first receptacle end 78 defining the first opening 82 may be symmetrical about a plane 90 bisecting the first receptacle end 78 and the first opening 82. The second receptacle end 80 defining the second opening 84 may also be symmetrical about a plane 90 bisecting the second receptacle end 80 and the second opening 84. It is to be appreciated that the plane 90 bisecting the first receptacle end 78 and the plane 90 bisecting the second receptacle end 80 may be the same. However, the planes 90 bisecting the first and second receptacle ends 78, 80 and the first and second openings 82, 84 need not be the same if, for example, the multipositional receptacle 76 bends, angles, or curves between the first and second receptacle ends 78, 80. Moreover, the multipositional receptacle 76 may be further defined as a bipositional receptacle 76 such that the tracker body 68 is couplable to the mount assembly 72 in only the first position and the second position.

Engagement of the first connector 70 and the second connector 74 may secure the tracker body 68 and the mount assembly 72 together. The tracker body 68 and the mount assembly 72 may not be unsecured (e.g., moved away from one another) without first disengaging the first connector 70 and the second connector 74. Some relative movement may be permitted while the tracker body 68 and the mount assembly 72 are secured together. Engagement of the first connector 70 and the second connector 74 may also fix a position of the tracker body 68 relative to the mount assembly 72. Said differently, engagement of the first connector 70 and the second connector 74 may prevent any movement of the tracker body 68 relative to the mount assembly 72 other than that caused by vibration.

Although not required, the first connector 70 may be integral with the tracker body 68. The first connector 70 may be formed integrally with the tracker body 68, such as by casting or molding (e.g., injection molding). Alternatively, the first connector 70 may be formed separately from the tracker body 68 and later joined with the tracker body 68, such as by welding (e.g. ultrasonic welding) or heat-staking to become integral.

As shown in FIGS. 3A-7, the tracker body 68 may have a hub 92, a first arm 94 extending away from the hub 92, a second arm 96 extending away from the hub 92, and a third arm 98 extending away from the hub 92. The first connector 70 may extend away from the hub 92, and the first connector 70 may extend transverse to the first arm 94, the second arm 96, and the third arm 98. The hub 92, the first arm 94, the second arm 96, and the third arm 98 may collectively present a first surface 100 facing in a first direction DR1, and the first connector 70 may extend away from the hub 92 in a second direction DR2 opposite the first direction DR1. The surgical tracker assembly 58 may further include a first fiducial marker 102 adjacent to a distal end 104 of the first arm 94, a second fiducial marker 110 adjacent to a distal end 106 of the second arm 96, and a third fiducial marker 112 adjacent to a distal end 108 of the third arm 98.

The first arm 94 extends a first distance D1 from the hub 92, the second arm 96 extends a second distance D2 from the hub, and the third arm 98 extends a third distance D3 from the hub 92. The first distance D1 may be greater than either of the second distance D2 and the third distance D3. In other words, the first distance D1 may be greater than the second distance D2, and the first distance D1 may be greater than the third distance D3. Engagement of the first connector 70 and the second connector 74 may orient the first arm 94 to extend toward the object to be tracked. Orientation of the first arm 94, i.e., the longest arm, toward the object to be tracked increases the accuracy of tracking the object.

Although the first arm 94, the second arm 96, and the third arm 98 are shown throughout the Figures, the tracker body 68 may have at least three arms. The tracker body 68 may further have a fourth arm extending away from the hub 92 and a fifth arm extending away from the hub 92. The first connector 70 may extend away from the hub 92 transverse to the first arm 94, the second arm 96, the third arm 98, the fourth arm, and the fifth arm. The fourth arm extends a fourth distance from the hub 92 and the fifth arm extends a fifth distance from the hub 92. The first distance D1 may be greater than any of the second distance, the third distance, the fourth distance, and the fifth distance. Engagement of the first connector 70 and the second connector 74 may orient the first arm 94 to extend toward the object to be tracked in the implementations with the first arm 94, the second arm 96, the third arm 98, the fourth arm, and the fifth arm.

The surgical tracker assembly 58 may further include a cover 114 couplable to the tracker body 68. The cover 114 may define a first window 116 adjacent to the first fiducial marker 102, a second window 118 adjacent to the second fiducial marker 110, and a third window 120 adjacent to the third fiducial marker 112. The first fiducial marker 102 may be viewable through the first window 116 by the localizer 44 of the robotic surgical system 10, the second fiducial marker 110 may be viewable through the second window 118 by the localizer 44 of the robotic surgical system 10, and the third fiducial marker 112 may be viewable through the third window 120 may the localizer 44 of the robotic surgical system 10. The first fiducial marker 102, the second fiducial marker 110, and the third fiducial marker 112 may be retro-reflective to increase the accuracy of tracking, and the cover 114 may have an anti-reflective (e.g. matte) finish to further increase the accuracy of tracking. The anti-reflective finish may be accomplished with a rough surface texture.

The mount assembly 72 may further include a stem 122 extending between a first stem end 124 and a second stem end 126. The second connector 74 may be disposed at the first stem end 124 of the stem 122, and the second stem end 126 may be configured to attach relative to the object to be tracked. As non-limiting examples, the second stem end 126 may be configured to attach through a clamp or a screw, among other possibilities. The second stem end 126 can be a clamp like that shown in U.S. Patent Publication No. 2022/0257334 A1, filed on Feb. 16, 2021, entitled, “Clamp Assembly For Fixing a Navigation Tracker to a Portion of Bone,” hereby incorporated herein by reference. The object to be tracked may be the patient, including a bone of the patient, and more specifically a scapula of the patient, a pelvis of the patient, a hip of the patient, or a humerus of a patient, among other possibilities.

The first connector 70 may be plastic, and the second connector 74 may be metallic. As non-limiting examples, the plastic of the first connector 70 may be polycarbonate and the metal of the second connector 74 may be stainless steel. As such, it is to be appreciated that the second connector 74 is capable of being easily sterilized without deforming, and thus is durable and re-usable, and the first connector 70 may be inexpensive to produce and thus disposable.

The projection 86 may include a first orientation feature 128 and the multipositional receptacle 76 may include a second orientation feature 130 complementary to the first orientation feature 128 of the projection 86. Alignment of the first orientation feature 128 and the second orientation feature 130 orients the tracker body 68 relative to the mount assembly 72. Although not required, the first orientation feature 128 and the second orientation feature 130 may orient the first arm 94, i.e., the longest arm, toward the object to be tracked.

Alignment of the first orientation feature 128 and the second orientation feature 130 may orient the tracker body 68 relative to the mount assembly 72 in a predefined orientation during engagement of the first connector 70 and the second connector 74 in the first position, and alignment of the first orientation feature 128 and the second orientation feature 130 may orient the tracker body 68 relative to the mount assembly 72 in the predefined orientation during engagement of the first connector 70 and the second connector 74 in the second position. In other words, the first orientation feature 128 and the second orientation feature 130 may orient the tracker body 68 relative to the mount assembly 72 in the same predefined orientation regardless of if the first connector 70 and the second connector 74 are engaged in the first position or in the second position.

More specifically, the multipositional receptacle 76 may extend along an axis A1 such that the first receptacle end 78 is spaced from the second receptacle end 80 along the axis A1, and the first orientation feature 128 and the second orientation feature 130 may rotationally orient the tracker body 68 relative to the mount assembly 72 about the axis A1. Said differently, the first orientation feature 128 and the second orientation feature 130 may rotationally orient the tracker body 68 and the mount assembly 72 relative to the axis A1. In this way, the first orientation feature 128 and the second orientation feature 130 act as a poka-yoke to prevent misorientation of the tracker body 68 and the mount assembly 72. Moreover, at least one of the first receptacle end 78 and the second receptacle end 80 has a keyed receptacle geometry 132 that forms the second orientation feature 130. In other words, the first receptacle end 78 may have the keyed receptacle geometry 132, the second receptacle end 80 may have the keyed receptacle geometry 132, or both the first receptacle end 78 and the second receptacle end 80 may have the keyed receptacle geometry 132.

The keyed receptacle geometry 132 may include a curved portion 134 and a flat portion 136. The flat portion 136 may be distal to the object to be tracked and the curved portion 134 may be proximal to the object to be tracked. It is to be understood that the curved portion 134 and the flat portion 136 of the keyed receptacle geometry 132 may form a D-shape. Additionally, the curved portion 134 may have a stem receiver 138 configured to receive the first stem end 124 of the stem 122.

The projection 86 may have a keyed projection geometry 140 that forms the first orientation feature 128. The keyed projection geometry 140 is shaped to confirm to the keyed receptacle geometry 132 such that alignment of the keyed projection geometry 140 and the keyed receptacle geometry 132 orients the tracker body 68 relative to the mount assembly 72 in the predefined orientation. The keyed projection geometry 140 may include a flat portion 136 distal to the object to be tracked and a curved portion 134 proximal to the object to be tracked. It is to be understood that the curved portion 134 and the flat portion 136 of the keyed projection geometry 140 may form a D-shape.

As shown in FIGS. 3A-7, the projection 86 may include a first resilient arm 142 and a second resilient arm 144 spaced from the first resilient arm 142. The multipositional receptacle 76 may present an inner surface 146, and the first resilient arm 142 and the second resilient arm 144 may be configured to engage the inner surface 146 of the multipositional receptacle 76. Engagement of the first resilient arm 142 and the second resilient arm 144 with the inner surface 146 of the multipositional receptacle 76 may preload the projection 86 relative to the multipositional receptacle 76. The first resilient arm 142 and the second resilient arm 144 may also extend through the passageway 88 defined between the first receptacle end 78 and the second receptacle end 80.

The first resilient arm 142 may include a first lip 148 extending away from the second resilient arm 144 and configured to abut the multipositional receptacle 76 to retain the tracker body 68 to the mount assembly 72, and the second resilient arm 144 may include a second lip 150 extending away from the first resilient arm 142 and configured to abut the multipositional receptacle 76 to retain the tracker body 68 to the mount assembly 72. The first lip 148 may taper toward a distal end 152 of the first resilient arm 142 and the second lip 150 may taper toward a distal end 154 of the second resilient arm 144. Moreover, the first resilient arm 142 may include a non-tapered portion 156 disposed between the first lip 148 and the distal end 152 of the first resilient arm 142, and the second resilient arm 144 may include a non-tapered portion 158 disposed between the second lip 150 and the distal end 154 of the second resilient arm 144.

Furthermore, the multipositional receptacle 76 may include a first narrowed portion 160 alignable to abut the first lip 148 of the first resilient arm 142 and a second narrowed portion 162 alignable to abut the second lip 150 of the second resilient arm 144. The first receptacle end 78 includes a first receptacle end surface 164 facing away from the second receptacle end 80 and the second receptacle end 80 includes a second receptacle end surface 166 facing away from the first receptacle end 78. The first narrowed portion 160 may present a first narrowed surface 168 indented relative to one of the first receptacle end surface 164 and the second receptacle end surface 166. In other words, the first narrowed surface 168 may be indented relative to the first receptacle end surface 164 or may be indented relative to the second receptacle end surface 166. Moreover, the second narrowed portion 162 may present a second narrowed surface 170 indented relative to the one of the first receptacle end surface 164 and the second receptacle end surface 166. In other words, the second narrowed surface 170 may be indented relative to the first receptacle end surface 164 or may be indented relative to the second receptacle end surface 166. It is to be appreciated that the first narrowed surface 168 and the second narrowed surface 170 may be both indented relative to the first receptacle end surface 164, or may be both indented relative to the second receptacle end surface 166.

The first narrowed portion 160 may present a third narrowed surface 172 indented relative to the other of the first receptacle end surface 164 and the second receptacle end surface 166. In other words, in one implementation, the first narrowed portion 160 presents the first narrowed surface 168 indented relative to the first receptacle end surface 164 and presents the third narrowed surface 172 indented relative to the second receptacle end surface 166. The second narrowed portion 162 may present a fourth narrowed surface 174 indented relative to the other of the first receptacle end surface 164 and the second receptacle end surface 166. Said differently, in one implementation, the second narrowed portion 162 presents the second narrowed surface 170 indented relative to the first receptacle end surface 164 and presents the fourth narrowed surface 174 indented relative to the second receptacle end surface 166.

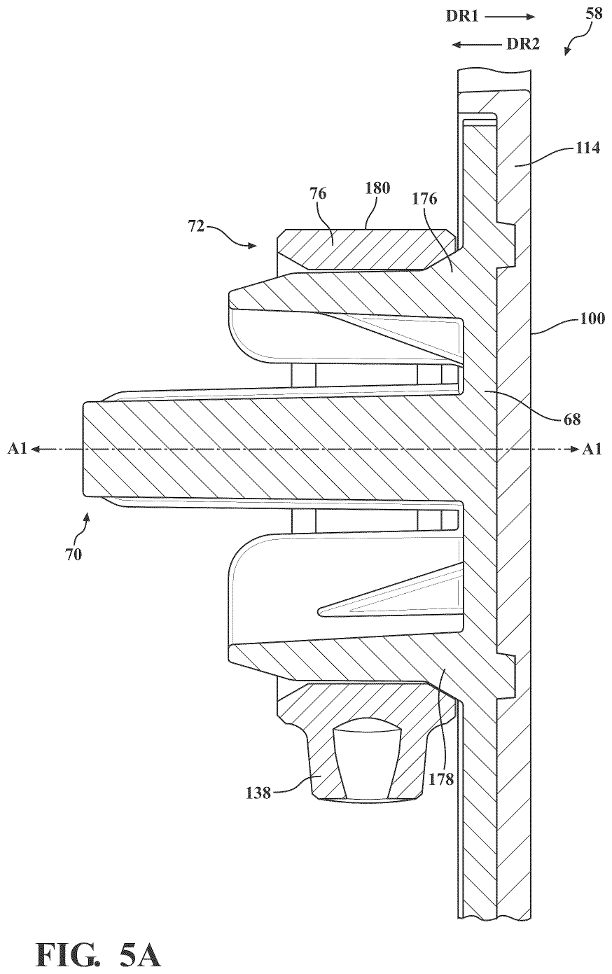

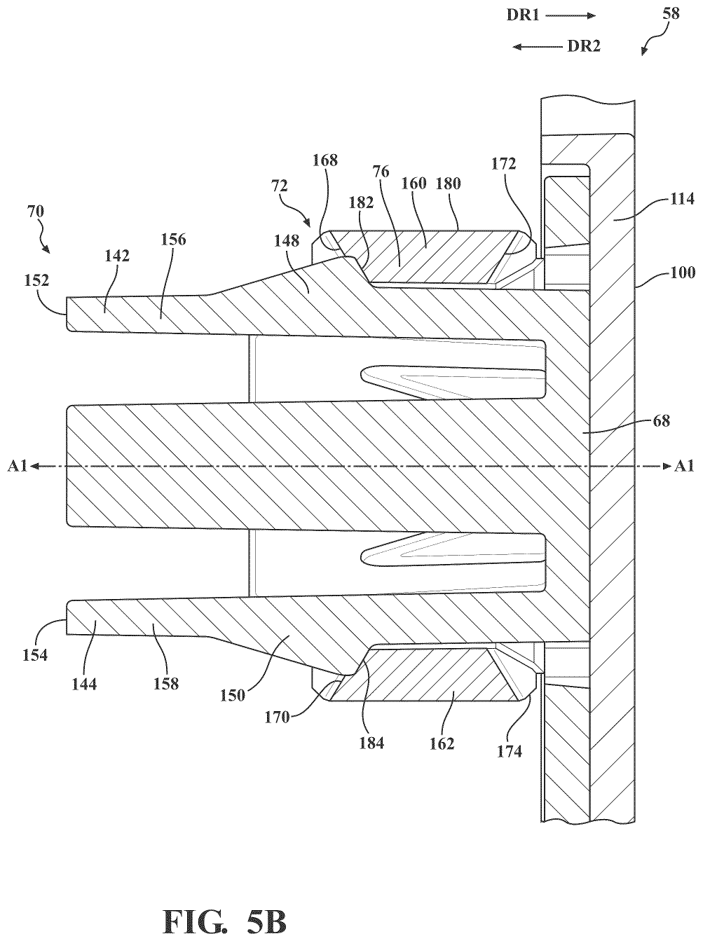

Although not required, as shown in FIG. 5A, the tracker body 68 may include a first stabilizing projection 176 extending toward the multipositional receptacle 76 and shaped to correspond to the multipositional receptacle 76. Additionally, the tracker body 68 may include a second stabilizing projection 178 extending toward the multipositional receptacle 76 and shaped to correspond to the multipositional receptacle 76. The first stabilizing projection 176 and the second stabilizing projection 178 may be circumferentially spaced relative to the first resilient arm 142 and the second resilient arm 144. Moreover, the first stabilizing projection 176 and the second stabilizing projection 178 may taper toward the multipositional receptacle 76. As such, the first stabilizing projection 176 and the second stabilizing projection 178 may be chamfers. The first stabilizing projection 176 and the second stabilizing projection 178 reduce vibration and micromotion between the tracker body 68 and the multipositional receptacle 76, thus increasing the accuracy of tracking. Moreover, the first stabilizing projection 176 and the second stabilizing projection 178 assist in centering the tracker body 68 relative to the multipositional receptacle 76, further increasing the accuracy of tracking, and the first stabilizing projection 176 and the second stabilizing projection 178 also reduce the relative tolerances required between the tracker body 68 and the multipositional receptacle 76 while still achieving the advantages described herein.

As shown in FIG. 5B, the multipositional receptacle 76 may extend along an axis A1 such that the first receptacle end 78 is spaced from the second receptacle end 80 along the axis A1. The multipositional receptacle 76 presents an outer surface 180 facing away from the axis A1, and the first narrowed surface 168 may extend between the inner surface 146 of the multipositional receptacle 76 and the outer surface 180 of the multipositional receptacle 76. The second narrowed surface 170 may extend between the inner surface 146 of the multipositional receptacle 76 and the outer surface 180 of the multipositional receptacle 76.

In one implementation, the first narrowed surface 168 extends perpendicularly relative to the axis A1, and the second narrowed surface 170 extends perpendicularly relative to the axis A1. In another implementation, the first narrowed surface 168 extends angularly offset relative to perpendicular to the axis A1, and the second narrowed surface 170 extends angularly offset relative to perpendicular to the axis A1. Moreover, in one implementation, the first narrowed surface 168 tapers toward the inner surface 146 of the multipositional receptacle 76, and the second narrowed surface 170 tapers toward the inner surface 146 of the multipositional receptacle 76. In another implementation, the first narrowed surface 168 tapers toward the outer surface 180 of the multipositional receptacle 76, and the second narrowed surface 170 tapers toward the outer surface 180 of the multipositional receptacle 76.

The first lip 148 may include a first abutment surface 182 shaped to correspond to the first narrowed surface 168, and the second lip 150 may include a second abutment surface 184 shaped to correspond to the second narrowed surface 170. Moreover, in the implementations where the first narrowed surface 168 and the second narrowed surface 170 taper, the first abutment surface 182 may have a corresponding taper to that of the first narrowed surface 168 and the second abutment surface 184 may have a corresponding taper to that of the second narrowed surface 170. The first abutment surface 182 and the second abutment surface 184 reduce vibration and micromotion between the tracker body 68 and the multipositional receptacle 76, thus increasing the accuracy of tracking. Moreover, the first abutment surface 182 and the second abutment surface 184 assist in centering the tracker body 68 relative to the multipositional receptacle 76, further increasing the accuracy of tracking, and the first abutment surface 182 and the second abutment surface 184 also reduce the relative tolerances required between the tracker body 68 and the multipositional receptacle 76 while still achieving the advantages described herein.

The first resilient arm 142 may be moveable toward the second resilient arm 144 and the second resilient arm 144 may be moveable toward the first resilient arm 142 to permit the projection 86 to be disposed in at least one of the first opening 82 and the second opening 84 of the multipositional receptacle 76. As described herein, the projection 86 may be disposed in the first opening 82, the projection 86 may be disposed in the second opening 84, or the projection 86 may be disposed in both the first opening 82 and the second opening 84. The first resilient arm 142 and the second resilient arm 144 may be configured to engage the multipositional receptacle 76 through a snap-fit.

In the implementations where the first resilient arm 142 and the second resilient arm 144 are configured to engage the multipositional receptacle 76 through the snap-fit, the multipositional receptacle 76 may be held by a medical professional and engaged with the first resilient arm 142 and the second resilient arm 144 through the snap-fit while the tracker body 68 is still within a sterile packaging surrounding the tracker body 68. More specifically, the medical professional may open the sterile packaging surrounding the tracker body 68, particularly near the first resilient arm 142 and the second resilient arm 144, and snap the multipositional receptacle 76 into engagement with the first resilient arm 142 and the second resilient arm 144. The medical professional may then remove the tracker body 68 from the sterile packaging without ever having physically hand-touched the tracker body 68. As such, by so doing, the medical professional thus has reduced the risk of touching the first fiducial marker 102, the second fiducial marker 110, or the third fiducial marker 112, which may transfer contaminants (e.g., oil from the hand, dirt, etc.) to the first fiducial marker 102 the second fiducial marker 110, or the third fiducial marker 112, which reduces the accuracy of tracking. Moreover, in so doing, the medical professional will also have maintained the sterility of the tracker body 68, reducing the risk of infection during a surgical procedure and thus increasing the safety of the surgical procedure.

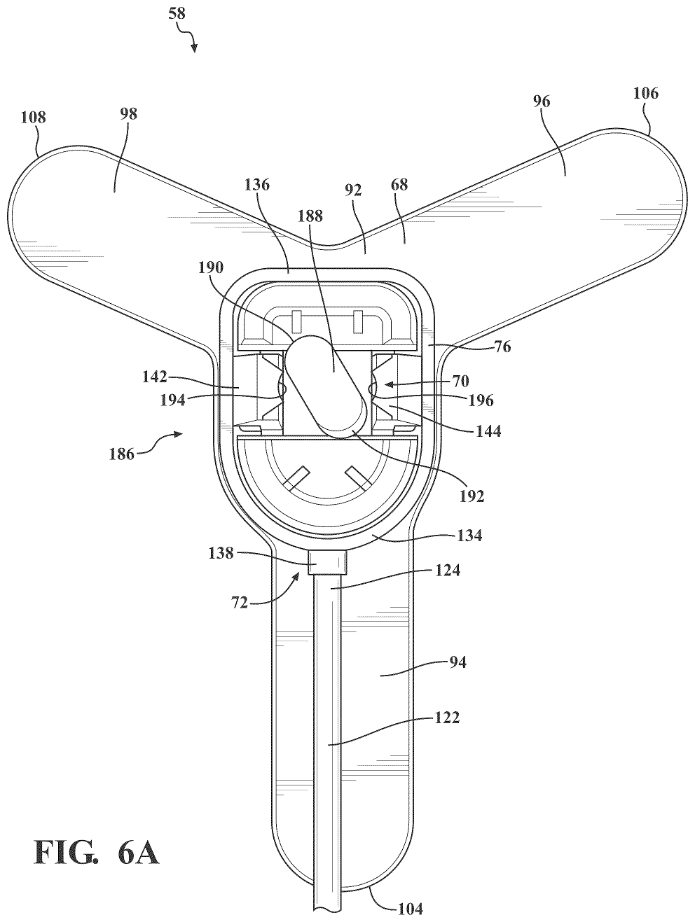

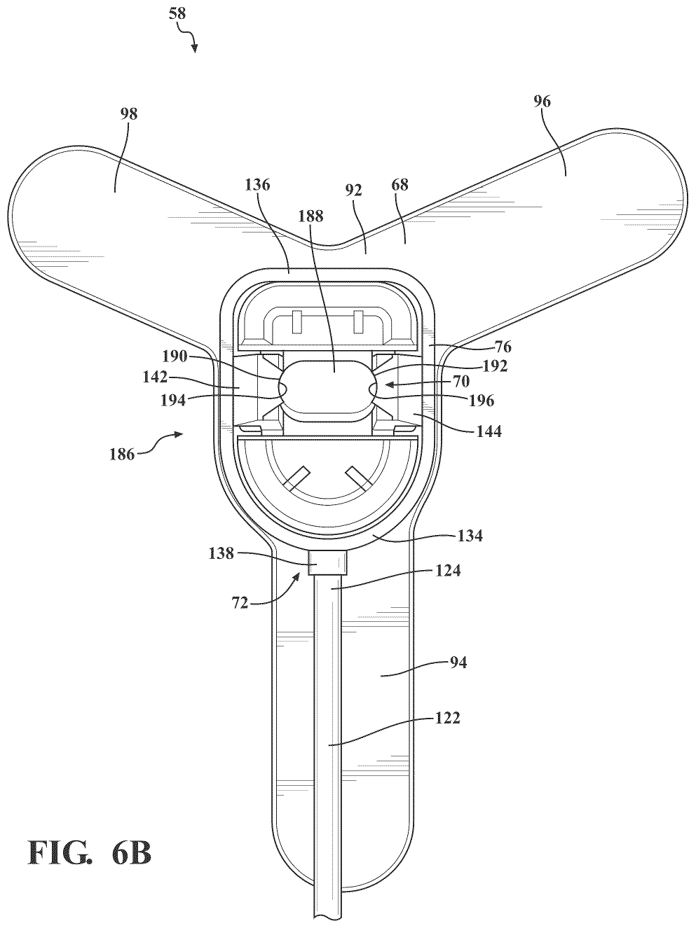

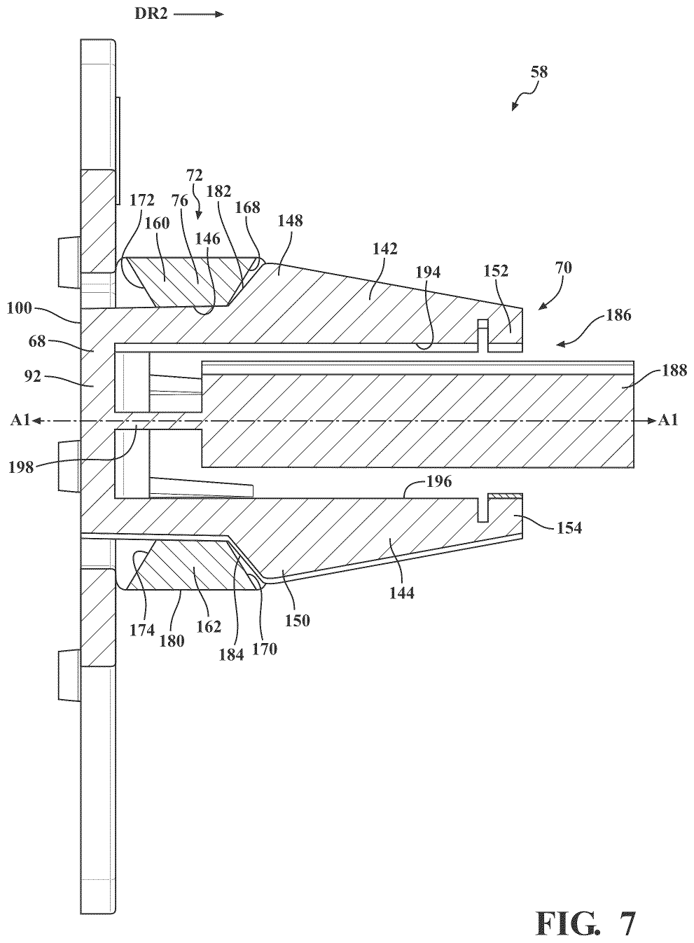

As shown in FIGS. 6A-7, the surgical tracker assembly 58 may further include a locking mechanism 186 couplable to the tracker body 68. The locking mechanism 186 is configured to secure the tracker body 68 to the mount assembly 72. In one implementation, the locking mechanism 186 includes a stop 188 disposed between the first resilient arm 142 and the second resilient arm 144. The stop 188 is configured to limit movement of the first resilient arm 142 and the second resilient arm 144 toward one another. The stop 188 may extend from the tracker body 68.

The stop 188 may be moveable between a first stop position, as shown in FIG. 6A, and a second stop position, as shown in FIG. 6B. More specifically, the stop 188 may be rotatable between the first stop position and the second stop position. In the first stop position, the first resilient arm 142 and the second resilient arm 144 are permitted to move toward one another. In the second stop position, the first resilient arm 142 and the second resilient arm 144 are limited from moving toward one another. It is to be appreciated that the first resilient arm 142 and the second resilient arm 144 may be prevented from moving toward one another whatsoever when the stop 188 is in the second stop position. Moreover, the stop 188 may be configured to bias the first resilient arm 142 and the second resilient arm 144 away from one another in the second stop position. Said differently, the stop 188 may be configured to move the first resilient arm 142 away from the second resilient arm 144, and may be configured to move the second resilient arm 144 away from the first resilient arm 142.

The stop 188 may have a first stop engagement surface 190 and a second stop engagement surface 192 facing away from the first stop engagement surface 190. The first resilient arm 142 may have a first inner arm surface 194 shaped to correspond to the first stop engagement surface 190, and the second resilient arm 144 may have a second inner arm surface 196 shaped to correspond to the second stop engagement surface 192. In one implementation, the first stop engagement surface 190 is convex, the second stop engagement surface 192 is convex, the first inner arm surface 194 is concave, and the second inner arm surface 196 is concave. In another implementation, the first stop engagement surface 190 is concave, the second stop engagement surface 192 is concave, the first inner arm surface 194 is convex, and the second inner arm surface 196 is convex. Other shapes of the first stop engagement surface 190, the second stop engagement surface 192, the first inner arm surface 194, and the second inner arm surface 196 are contemplated, such as but not limited to polygonal shapes.

The stop 188 may include a weakened region 198. The stop 188 is breakable at the weakened region 198. The weakened region 198 may have a reduced diameter relative to adjacent portions of the stop 188, as shown in FIG. 7. The weakened region 198 may also define perforations, channels, or other voids which aid in the stop 188 being breakable at the weakened region 198. The weakened region 198 may further be comprised of a material that is more susceptible to break relative to the material of adjacent portions of the stop 188, particularly in response to rotation of the stop 188 between the first stop position and the second stop position. Additionally, the weakened region 198 of the stop 188 may be proximal to the tracker body 68.

The stop 188 may be configured to break at the weakened region 198 in response to torsional shear stress on the weakened region 198. The torsional shear stress on the weakened region 198 may result from rotation of the stop 188. More specifically, the stop 188 may be configured to break at the weakened region 198 in response to rotation of the stop 188 from the first stop position to the second stop position. Said differently, the stop 188 may break at the weakened region 198 during movement of the stop 188 from the first stop position toward the second stop position. Although not required, the stop 188 may be configured to remain disposed between the first resilient arm 142 and the second resilient arm 144 upon breaking. More specifically, the stop 188 may be configured to remain disposed in the second stop position upon breaking. The weakened region 198 of the stop 188 may also provide an indicium of use upon breaking. A medical professional may view the broken stop and understand that the surgical tracker assembly 58 has already been used. Moreover, the tracker body 68 may be disposable. Thus, the indicium of use allows the medical professional to know if a tracker body 68 has already been used and needs to be disposed of. To this end, the tracker body 68 may include a single use labeling thereon. In a non-limiting example, the single use labeling is embossed on the tracker body 68.

Although not required, the locking mechanism 186 may be disposable through the first opening 82 of the first receptacle end 78 and the second opening 84 of the second receptacle end 80. The locking mechanism 186 may be accessible by hand (e.g., the hand of a medical professional) to secure the tracker body 68 to the mount assembly 72 upon engagement of the first connector 70 of the tracker body 68 and the second connector 74 of the mount assembly 72. In another implementation, the projection 86 includes a first magnet and the multipositional receptacle 76 includes a second magnet attractable to the first magnet. The first magnet and the second magnet further assist the tracker body 68 in being secured to the mount assembly 72.

In a second aspect, the surgical tracker assembly 58 for tracking an object includes a tracker body 68 and a mount assembly 72. The mount assembly 72 includes a first attachment portion 200 connectable to the tracker body 68 and a second attachment portion 202 connectable to the object. The first attachment portion 200 defines a first keyed geometry 204 and includes flexible connection members 206, 208. The second attachment portion 202 defines an aperture 210 configured to receive and connect with the flexible connection members 206, 208 of the first attachment portion 200 through either side of the aperture 210. The aperture 210 defined by the second keyed geometry 212 conforms to the first keyed geometry 204 such that the second attachment portion 202 receives the first attachment portion 200 in a predefined orientation.

In a third aspect, the surgical tracker assembly 58 includes a tracker body 68 having a back surface 214. The back surface 214 defines an attachment base 216. The surgical tracker assembly 58 also includes a first attachment mechanism 218 including a first keying feature 220 including flexures 222, 224 and tabs 226, 228. The first keying feature 220 has a shape of irregular geometry. The surgical tracker assembly 58 further includes a second attachment mechanism 230 including an open ring 232 including a second keying feature 234 which is configured to receive the first keying feature 220. The open ring 232 has a shape of irregular geometry complementary to the shape of the first keying feature 220. The second attachment mechanism 230 is configured to receive the first attachment mechanism 218 through either side of the open ring 232 and maintain a snap lock between the first attachment mechanism 218 and the second attachment mechanism 230. The attachment base 216 is rigidly coupled to one of the first attachment mechanism 218 and the second attachment mechanism 230.

In a fourth aspect, a mount assembly 72 for a surgical tracker assembly 58 is provided. The mount assembly 72 includes a first attachment base 236 connected to a tracker body 68, a second attachment base 238 connected to an object, a first attachment mechanism 218 including a first keying feature 220 including flexures 222, 224 and tabs 226, 228, and a second attachment mechanism 230 including an open ring 232. The open ring 232 includes a second keying feature 234 which is configured to receive the first keying feature 220. The second attachment mechanism 230 is configured to receive the first attachment mechanism 218 through either side 240, 242 of the open ring 232. The first attachment base 236 is rigidly coupled to one of the first attachment mechanism 218 and the second attachment mechanism 230. The second attachment base 238 is coupled to the other of the first attachment mechanism 218 and the second attachment mechanism 230.

In a fifth aspect, the surgical tracker assembly 58 for tracking an object includes a tracker body 68 and a mount assembly 72. The mount assembly 72 includes a first attachment portion 200 coupled to the tracker body 68 and including a connection member 244. The mount assembly 72 also includes a second attachment portion 202 couplable to the object, the second attachment portion 202 includes a receptacle 246 including an open ring 232 configured to receive the connection member 244 therethrough. The receptacle 246 is configured to receive and connect with the connection member 244 through either one of opposing sides 240, 242 of the open ring 232.

In a sixth aspect, a mount assembly 72 for a surgical tracker assembly 58 configured to track an object is provided. The mount assembly 72 includes a first attachment portion 200 coupled or connectable to a tracker body 68. The first attachment portion 200 includes a connection member 244. The mount assembly 72 also includes a second attachment portion 202 coupled or connectable to the object. The second attachment portion 202 includes a receptacle 246 including an open ring 232 configured to receive the connection member 244 therethrough. The receptacle 246 is configured to receive and connect with the connection member 244 through either one of opposing sides 240, 242 of the open ring 232.

In a seventh aspect, the surgical tracker assembly 58 for tracking an object includes a tracker body 68 and a mount assembly 72. The mount assembly 72 includes a first attachment portion 200 coupled or connectable to the object. The first attachment portion 200 includes a first connection member 248 extending in a first direction and a second connection member 250 extending in a second direction. The mount assembly 72 also includes a second attachment portion 202 coupled or connectable to the tracker body 68. The second attachment portion 202 includes a receptacle 246 including an open ring 232. The receptacle 246 is configured to receive and connect with either of the first or the second connection member 248, 250 through either one of opposing sides 240, 242 of the open ring 232.

In an eighth aspect, a mount assembly 72 for a surgical tracker assembly 58 configured to track an object is provided. The mount assembly 72 includes a first attachment portion 200 coupled or connectable to the object. The first attachment portion 200 includes a first connection member 248 extending in a first direction and a second connection member 250 extending in a second direction. The mount assembly 72 also includes a second attachment portion 202 coupled or connectable to a tracker body 68. The second attachment portion 202 includes a receptacle 246 including an open ring 232. The receptacle 246 is configured to receive and connect with either of the first or the second connection members 248, 250 through either one of opposing sides 240, 242 of the open ring 232.

In a ninth aspect, the surgical tracker assembly 58 for tracking a bone is provided. The surgical tracker assembly 58 includes a tracker body 68. The tracker body 68 includes three arms 94, 96, 98 extending in different directions from one another. Each arm 94, 96, 98 supports a fiducial marker 102, 110, 112 near a distal end 104, 106, 108 of each arm 94, 96, 98. One of the three arms 94, 96, 98 is a longest arm and includes a length that is greater than a length each of the other arms 94, 96, 98. The surgical tracker assembly 58 also includes a first attachment portion 200 coupled or connectable to the tracker body 68 and defining a first keyed geometry 204. The surgical tracker assembly 58 further includes a second attachment portion 202 coupled or connectable to the bone and defining a second keyed geometry 212 conforming to the first keyed geometry 204 such that the second attachment portion 202 receives the first attachment portion 200 in a predefined orientation. The predefined orientation causes the longest arm 94 of the tracker body 68 to be oriented in a direction toward the bone.

In a tenth aspect, the surgical tracker assembly 58 for tracking an object includes a tracker body 68 and a mount assembly 72. The mount assembly 72 includes a first attachment portion 200 coupled or connectable to the tracker body 68. The first attachment portion 200 includes a flexible connection member 244 and a locking mechanism 186 disposed adjacent to the flexible connection member 244. The mount assembly 72 also includes a second attachment portion 202 coupled or connectable to the object. The second attachment portion 202 includes a receptacle 246 including an open ring 232 configured to receive the flexible connection member 244 therethrough. The flexible connection member 244 couples to the receptacle 246 through a snap-fit, and the locking mechanism 186 is configured to be rotated to bias the flexible connection member 244 against the receptacle 246 to lock the flexible connection member 244 to the receptacle 246.

In an eleventh aspect, the surgical tracker assembly 58 for tracking an object includes a tracker body 68 and a mount assembly 72 couplable to the tracker body 68. The tracker body 68 includes a first connector 70. The mount assembly 72 also includes a second connector 74 engageable with the first connector 70 of the tracker body 68. The surgical tracker assembly 58 further includes a locking mechanism 186 couplable to the tracker body 68 and configured to secure the tracker body 68 to the mount assembly 72. One of the first connector 70 of the tracker body 68 and the second connector 74 of the mount assembly 72 includes a receptacle 246 extending between a first receptacle end 78 and a second receptacle end 80. The first receptacle end 78 defines a first opening 82 and the second receptacle end 80 defines a second opening 84. The other of the first connector 70 of the tracker body 68 and the second connector 74 of the mount assembly 72 includes a projection 86 disposable in at least one of the first opening 82 of the first receptacle end 78 and the second opening 84 of the second receptacle end 80 to couple the tracker body 68 to the mount assembly 72.

In a twelfth aspect, the surgical tracker assembly 58 includes a tracker body 68 and a mount assembly 72. The tracker body 68 includes a first connector 70 and the mount assembly 72 includes a second connector 74 releasably engageable with the first connector 70. One of the connectors 70, 74 is a rigid connector 252 and the other of the connectors 70, 74 is a flexible connector 254 configured to flex into engagement with the rigid connector 252. The rigid and flexible connectors 252, 254 are configured to couple to one another in at least two different poses.

In a thirteenth aspect, the surgical tracker assembly 58 for tracking an object includes the tracker body 68 including the first connector 70. The mount assembly 72 is couplable to the tracker body 68. The mount assembly 72 includes the second connector 74 engageable with the first connector 70 in at least a first position and a second position different from the first position. One of the first connector 70 and the second connector 74 is rigid, and the other of the first connector 70 and the second connector 74 is resilient.

In one implementation of the thirteenth aspect, the first connector 70 is rigid and the second connector 74 is resilient. In another implementation of the thirteenth aspect, the first connector 70 is resilient and the second connector 74 is rigid. The first connector 70 or the second connector 74 that is rigid may take several forms. As non-limiting examples, the first connector 70 or the second connector 74 that is rigid may be a rigid post, a rigid ring, a rigid sleeve, or a rigid groove. The first connector 70 or the second connector 74 that is flexible may take several forms. As non-limiting examples, the first connector 70 or the second connector 74 that is flexible may be a flexible post, a flexible ring, a flexible sleeve, or a flexible groove. The first connector 70 and the second connector 74 may engage one another in at least two different positions. Said differently, the first connector 70 and the second connector 74 may enable multipositional engagement, for example, bipositional engagement. As non-limiting examples, the rigid post, the rigid ring, the rigid sleeve, or the rigid groove may have two rigid ends, and the flexible post, the flexible ring, the flexible sleeve, or the flexible groove may flex into engagement with either of the two rigid ends. As other non-limiting examples, the flexible post, the flexible ring, the flexible sleeve, or the flexible groove may have two flexible ends, and the rigid post, the rigid ring, the rigid sleeve, or the rigid groove may cause either of the two flexible ends to flex into engagement with the rigid post, the rigid ring, the rigid sleeve, or the rigid groove.

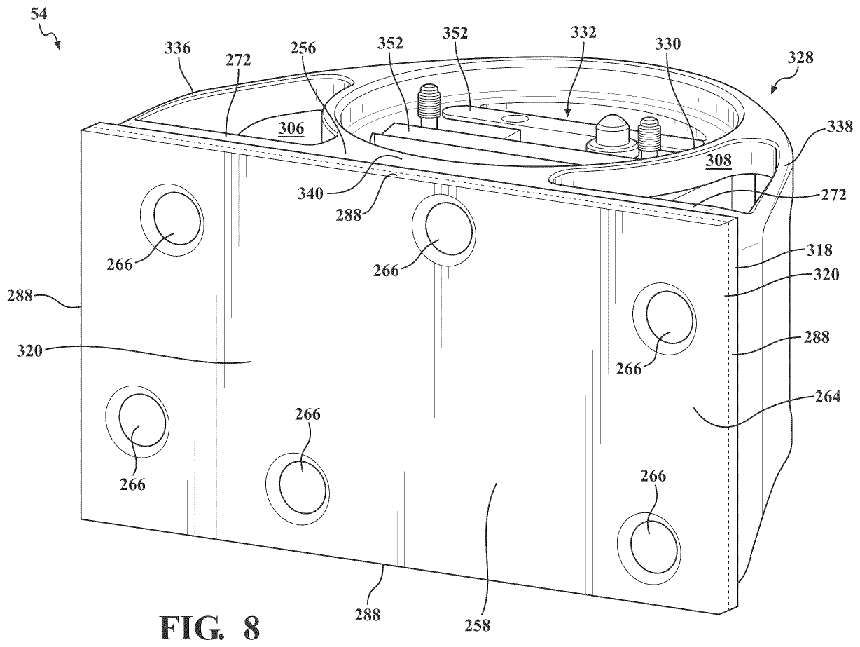

In a fourteenth aspect, as shown in FIGS. 8-10, a surgical tracker assembly 54 detectable by a localizer 44 is provided. The surgical tracker assembly 54 includes a support body 256 and a tracker plate 258. The support body 256 has an interface surface 260 defining an open receptacle 262. The tracker plate 258 includes a front side 264 supporting a plurality of tracking elements 266 detectable by the localizer 44. The tracker plate 258 also includes a rear side 268 facing away from the front side 264. The rear side 268 includes a connector 270 disposable in the open receptacle 262 to couple the tracker plate 258 to the interface surface 260 of the support body 256. The connector 270 may be removeable from the open receptacle 262 such that the tracker plate 258 may be decoupled from the interface surface 260 of the support body 256.

The tracker plate 258 having the front side 264 supporting the plurality of tracking elements 266 and the rear side 268 of the tracker plate 258 having a connector 270 disposable in the open receptacle 262 of the interface surface 260 of the support body 256 permits the tracker plate 258 to be coupled, and subsequently decoupled, from the interface surface 260 of the support body 256. As such, the tracker plate 258 may be disposed of after use. The tracker plate 258 being disposable is advantageous because contaminants (e.g., oil from the hand, dirt, etc.) may be transferred to the plurality of tracking elements 266, which reduces the accuracy of tracking. Moreover, the tracker plate 258 being disposable also have assists in maintaining the sterility of the surgical tracker assembly 54, reducing the risk of infection during a surgical procedure and thus increasing the safety of the surgical procedure.

Although not required, the support body 256 may include a support plate 272 extending approximately parallel to the tracker plate 258. The support plate 272 has the interface surface 260 defining the open receptacle 262. The tracker plate 258 may be substantially planar. In other words, the tracker plate 258 may extend substantially along a plane or may be substantially flat. Moreover, the support plate 272 may also be substantially planar. In other words, the support plate 272 may extend substantially along a plane or may be substantially flat.

The connector 270 may be further defined as a resilient arm 274. The resilient arm 274 may have a tab 276 extending from a distal end 278 of the resilient arm 274, and the tab 276 is engageable with the interface surface 260 of the support body 256. The connector 270 may be engageable with the support body 256 through a snap-fit. Additionally or alternatively, the connector 270 may include a first magnet and the interface surface 260 adjacent to the open receptacle 262 may include a second magnet attractable to the first magnet.

The rear side 268 of the tracker plate 258 may further include a locating wall 280 disposable in the open receptacle 262. The locating wall 280 is rigidly affixed relative to the tracker plate 258. The connector 270 and the locating wall 280 may be disposed opposite one another when disposed in the open receptacle 262. The connector 270 may be configured to bias the locating wall 280 against the support body 256 in a first direction DR1. The locating wall 280 both assists in positioning the tracker plate 258 relative to the interface surface 260 and also maintaining the position of the tracker plate 258 relative to the interface surface 260 once positioned, thus increasing the accuracy of tracking which is affected by vibration or micromotion.

The connector 270 may be further defined as a first connector 270. The rear side 268 of the tracker plate 258 may further include a second connector 282 disposable in the open receptacle 262 to couple the tracker plate 258 to the interface surface 260 of the support body 256. The second connector 282 may be further defined as a second resilient arm 284 which may include any combination of characteristics of the resilient arm 274 as described herein. The second connector 282 may be arranged circumferentially spaced from the first connector 270 relative to the open receptacle 262. The rear side 268 of the tracker plate 258 may further include a second locating wall 286 disposable in the open receptacle 262, and the second locating wall 286 is rigidly affixed relative to the tracker plate 258. The second locating wall 286 both assists in positioning the tracker plate 258 relative to the interface surface 260 and also maintaining the position of the tracker plate 258 relative to the interface surface 260 once positioned, thus increasing the accuracy of tracking which is affected by vibration or micromotion.

The second connector 282 and the second locating wall 286 may be disposed opposite one another when disposed in the open receptacle 262. The second connector 282 may be configured to bias the second locating wall 286 against the support body 256 in a second direction DR2 circumferentially spaced from the first direction DR1. As non-limiting examples, the second direction DR2 may be offset from the first direction by between about 10 degrees and about 180 degrees, by between about 20 degrees and about 180 degrees, by between about 30 degrees and about 180 degrees, by about 40 degrees and about 180 degrees, by between about 50 degrees and about 180 degrees, by between about 60 degrees and about 180 degrees by between about 70 degrees and about 180 degrees, by between about 80 degrees and about 180 degrees, by between about 90 degrees and about 180 degrees, by between about 10 degrees and about 90 degrees, by between about 20 degrees and about 90 degrees, by between about 30 degrees and about 90 degrees, by between about 40 degrees and about 90 degrees, by between about 50 degrees and about 90 degrees, by between about 60 degrees and about 90 degrees, by between about 70 degrees and about 90 degrees, by between about 80 degrees and about 90 degrees, and by about 90 degrees.

The rear side 268 of the tracker plate 258 may include a perimeter 288, and the first connector 270 may be circumferentially spaced from the second connector 282 relative to the perimeter 288 of the rear side 268 of the tracker plate 258. As a non-limiting example, the first connector 270 may be circumferentially spaced from the second connector 282 relative to the perimeter 288 of the rear side 268 between about 10 degrees and about 180 degrees, by between about 20 degrees and about 180 degrees, by between about 30 degrees and about 180 degrees, by about 40 degrees and about 180 degrees, by between about 50 degrees and about 180 degrees, by between about 60 degrees and about 180 degrees by between about 70 degrees and about 180 degrees, by between about 80 degrees and about 180 degrees, by between about 90 degrees and about 180 degrees, by between about 10 degrees and about 90 degrees, by between about 20 degrees and about 90 degrees, by between about 30 degrees and about 90 degrees, by between about 40 degrees and about 90 degrees, by between about 50 degrees and about 90 degrees, by between about 60 degrees and about 90 degrees, by between about 70 degrees and about 90 degrees, by between about 80 degrees and about 90 degrees, and by about 90 degrees.

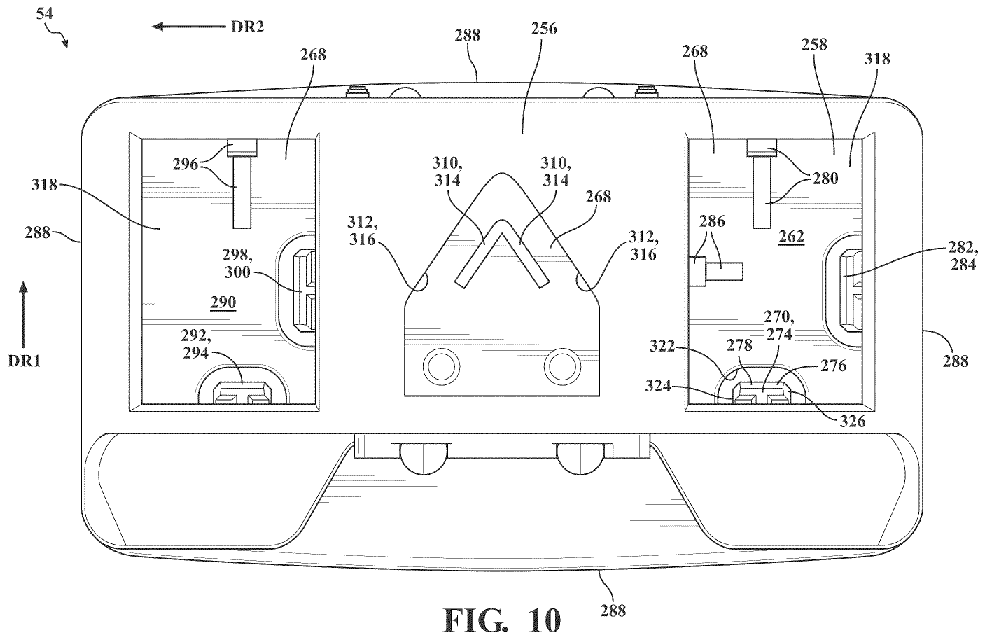

Although not required, as shown in FIG. 10, the interface surface 260 of the support body 256 may define a second open receptacle 290. The rear side 268 of the tracker plate 258 may include a third connector 292 disposable in the second open receptacle 290 to couple the tracker plate 258 to the interface surface 260 of the support body 256. The third connector 292 may be formed as a third resilient arm 294 extending away from the rear side 268. The third resilient arm 294 may include any combination of characteristics of the resilient arm 274 as described herein. The rear side 268 of the tracker plate 258 may further include a third locating wall 296 disposable in the second open receptacle 290. The third locating wall 296 is rigidly affixed relative to the tracker plate 258. More specifically, the third locating wall 296 may be rigidly affixed to the rear side 268 of the tracker plate 258. The third connector 292 and the third locating wall 296 may be disposed opposite one another when disposed in the second open receptacle 290. The third locating wall 296 both assists in positioning the tracker plate 258 relative to the interface surface 260 and also maintaining the position of the tracker plate 258 relative to the interface surface 260 once positioned, thus increasing the accuracy of tracking which is affected by vibration or micromotion.

The third connector 292 may be configured to bias the third locating wall 296 against the support body 256 in the first direction DR1. The first locating wall 280 and the third locating wall 296 may together approximate a line. More specifically, the first locating wall 280 and the third locating wall 296 may together approximate a line of contact between the tracker plate 258 and the support body 256. As such, the first locating wall 280 and the third locating wall 296 may cooperate to together reduce vibration or micromotion.

The rear side 268 of the tracker plate 258 may include a fourth connector 298 disposable in the second open receptacle 290 to couple the tracker plate 258 to the interface surface 260 of the support body 256. The fourth connector 298 may be formed as a fourth resilient arm 300 extending away from the rear side 268. The fourth resilient arm 300 may include any combination of characteristics of the resilient arm 274 as described herein. The rear side 268 of the tracker plate 258 may be free of a fourth locating wall disposable in the second open receptacle 290 opposite the fourth connector 298. The tracker plate 258 being free of a fourth locating wall assists in removing the tracker plate 258 from the interface surface 260 of the support body 256. More specifically, in the implementations where the tracker plate 258 has the second locating wall 286 but is free of the fourth locating wall, the second locating wall 286 may approximate only a point of contact and may not approximate a line of contact, thus permitting some relative movement between the tracker plate 258 and the interface surface 260 to aid in removing the tracker plate 258 from the interface surface 260.

Alternatively, it is to be appreciated that the rear side 268 of the tracker plate 258 may include a fourth locating wall disposable in the second open receptacle 290. In the implementations with the fourth locating wall, the fourth locating wall is rigidly affixed relative to the tracker plate 258. Moreover, in the implementations with the fourth locating wall, the fourth connector 298 and the fourth locating wall may be disposed opposite one another when disposed in the second open receptacle 290. In the implementations with the fourth locating wall, the fourth connector 298 may be configured to bias the fourth locating wall against the support body 256 in the second direction DR2. However, in the implementations where the rear side 268 of the tracker plate 258 is free of a fourth locating wall, the fourth connector 298 may still be configured to bias the tracker plate 258 in the second direction DR2.

The connector 270 may be integral with the rear side 268 of the tracker plate 258. Additionally, the second connector 282 may be integral with the rear side 268 of the tracker plate 258, the third connector 292 may be integral with the rear side 268 of the tracker plate 258, and the fourth connector 298 may be integral with the rear side 268 of the tracker plate 258. The first connector 270, the second connector 282, the third connector 292, and/or the fourth connector 298 may be formed integrally with the rear side 268 of the tracker plate 258, such as by casting or molding (e.g., injection molding). Alternatively, the first connector 270, the second connector 282, the third connector 292, and/or the fourth connector 298 may be formed separately from the rear side of the tracker plate 258 and later joined with the rear side 268 of the tracker plate 258, such as by welding (e.g. ultrasonic welding) or heat-staking to become integral.

The first locating wall 280 may be integral with the rear side 268 of the tracker plate 258. Additionally, the second locating wall 286 may be integral with the rear side 268 of the tracker plate 258, the third locating wall 296 may be integral with the rear side 268 of the tracker plate 258, and in the implementations with the fourth locating wall, the fourth locating wall may be integral with the rear side 268 of the tracker plate 258. The first locating wall 280, the second locating wall 286, the third locating wall 296, and/or the fourth locating wall may be formed integrally with the rear side 268 of the tracker plate 258, such as by casting or molding (e.g., injection molding). Alternatively, the first locating wall 280, the second locating wall 286, the third locating wall 296, and/or the fourth locating wall may be formed separately from the rear side of the tracker plate 258 and later joined with the rear side 268 of the tracker plate 258, such as by welding (e.g. ultrasonic welding) or heat-staking to become integral.

The tracker plate 258 may further include a stand-off 302 extending away from the rear side 268 and configured to ensure a gap 304 is defined between the rear side 268 and an adjacent object. More specifically, the stand-off may extend toward and be contactable with the support body 256 to ensure the gap 304 is defined the tracker plate 258 and the support body 256. The stand-off 302 may be further defined as a plurality of stand-offs 302 extending away from the rear side 268 and configured to ensure the gap 304 is defined between the rear side 268 and the adjacent object. More specifically, the stand-off 302 may be further defined as a plurality of stand-offs 302 extending toward and contactable the support body 256 to ensure the gap 304 is defined between the tracker plate 258 and the support body 256. As non-limiting examples, the plurality of stand-offs 302 may include two stand-offs 302, three stand-offs 302, four stand-offs 302, five stand-offs 302, six stand-offs 302, seven stand-offs 302, eight stand-offs 302, nine stand-offs 302, ten stand-offs 302, or more than ten stand-offs 302. The gap 304 ensured by the stand-off 302, or the plurality of stand-offs 302, assists in tracking the support body 256 through ensuring consistent relative spatial relation between the support body 256 and the plurality of tracking elements 266.

The tracker plate 258 may further include a first orientation feature 310 and the support body 256 may further include a second orientation feature 312 complementary to the first orientation feature 310 of the tracker plate 258. Alignment of the first orientation feature 310 and the second orientation feature 312 orients the tracker plate 258 relative to the support body 256 in a predefined orientation. Although not required, as shown in FIG. 10, the tracker plate 258 may include a keyed projection 314 that forms the first orientation feature 310, and the support body 256 defines a keyed receptacle 316 that forms the second orientation feature 312. The keyed projection 314 of the tracker plate 258 is disposable at least partially in the keyed receptacle 316 of the support body 256 to orient the tracker plate 258 relative to the support body 256. The tracker plate 258 may further include one or more additional projections which act as orientation feature(s), or poke-yoke features, such as but not limited to a first poke-yoke projection extending from the tracker plate 258 and into the keyed receptacle 316 and a second poke-yoke projection extending from the tracker plate 258 and into the keyed receptacle 316. The first poke-yoke projection and the second poke-yoke projection are shown in FIG. 10 and assist in orienting the tracker plate 258 relative to the support body 256 in the predefined orientation.

The support body 256 may be comprised of a first material and the tracker plate 258 may be comprised of a second material different from the first material. The support body 256 may be metallic, such as but not limited to stainless steel, and the tracker plate 258 may be plastic, such as but not limited to polycarbonate. As such, the rear side 268 may be plastic. The support body 256 being metallic permits the support body 256 to be sterilized, and thus re-used. The support body 256 may be cast in the implementations where the support body 256 is metallic. The tracker plate 258 being plastic permits the tracker plate 258 to be inexpensive, and thus disposable. The tracker plate 258 may be molded (e.g., injection molded) in the implementations where the tracker plate 258 is plastic. It is to be appreciated that the tracker plate 258 may be detachable from the support body 256.

In another implementation, the support body 256 may be comprised of the first material and the tracker plate 258 may be comprised of the same first material as the support body 256. In this way, the tracker plate 258 may be metallic, such as but not limited to stainless steel, permitting the tracker plate 258 to be sterilized, and thus re-used. The tracker plate 258 may be cast in the implementations where the tracker plate 258 is metallic.

Although not required, the tracker plate 258 may include a backing plate 318 having the rear side 268 and may include a face plate 320 having the front side 264. The face plate 320 and the backing plate 318 are affixed together. As non-limiting examples, the backing plate 318 and the face plate 320 may be affixed together through at least one chosen from heat staking and ultrasonic welding. It is also to be appreciated that the backing plate 318 and the face plate 320 may also be affixed together through mechanical fasteners. The single use labelling may be on the face plate 320, may be on the backing plate 318, or may be on both the face plate 320 and the backing plate 318. In a non-limiting example, the single use labelling may be embossed on the face plate 320, may be embossed on the backing plate 318, or may be embossed on both the face plate 320 and the backing plate 318.

Moreover, the backing plate 318 may define a connector aperture 322. The face plate 320 may prevent the connector aperture 322 and the connector 270 from being viewable from the front side 264. The connector 270 may include a parallel portion 324 extending parallel to the tracker plate 258 and into the connector aperture 322. The connector 270 may also include a perpendicular portion 326 extending from the parallel portion 324 away from the backing plate 318. The perpendicular portion 326 and the parallel portion 324 together permit the connector 270 to bend in two separate directions. More specifically, the perpendicular portion 326 may extend from the parallel portion 324 along an axis which is angularly offset from an axis perpendicular to a plane along which the tracker plate 258, and thus also the rear side 268 of the tracker plate 258, extends. In the implementations with the locating wall 280, the connector 270 may be angularly offset to extend away from the locating wall 280 when the tracker plate 258 is detached from the support body 256. Thus, when the tracker plate 258 is attached to the support body 256, the connector 270 is forced toward the locating wall 280 to bias the locating wall 280 against the support body 256.

The plurality of tracking elements 266 may be further defined as at least six tracking elements 266 detectable by the localizer 44. The plurality of tracking elements 266 may be retro-reflective. Additionally, the front side 264 of the tracker plate 258 may have an anti-reflective (e.g. matte) finish. The anti-reflective finish may be accomplished with a rough surface texture.

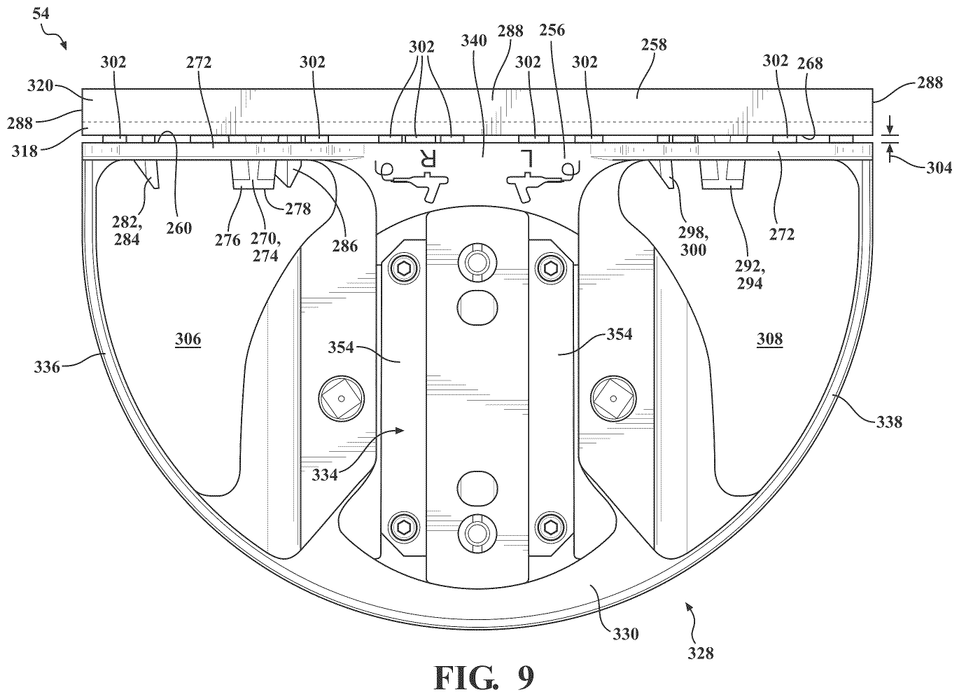

As shown in FIGS. 8 and 9, a coupler 328 includes the surgical tracker assembly 54 and a coupler body 330 configured to couple to the support body 256 of the surgical tracker assembly 54. The coupler body 330 may include a first coupling interface 332 configured to connect to the robotic manipulator 14 of the robotic surgical system 10, and the coupler body 330 may include a second coupling interface 334 facing away from the first coupling interface 332 and configured to connect to the tool 26 of the robotic surgical system 10. An axis may extend between the first coupling interface 332 and the second coupling interface 334, and the coupler body 330 may extend circumferentially about the axis such that the coupler 328 is generally cylindrical.

As shown in FIG. 9, the support body 256 may include a first support arm 336 extending away from the support plate 272, the first support arm 336 may be configured to couple to the coupler 328, and the first support arm 336 may extend to the coupler body 330. The support body 256 may also include a second support arm 338 extending away from the support plate 272, the second support arm 338 may be spaced from the first support arm 336, the second support arm 338 may be configured to couple to the coupler 328, and the second support arm 338 may extend to the coupler body 330.

Although not required, the support body 256 may be integral with the coupler body 330. As such, in the implementations with the first support arm 336 and the second support arm 338, the first support arm 336 and the second support arm 338 may be integral with the coupler body 330. The support body 256 may be formed integrally with the coupler body 330, such as by casting. Alternatively, the support body 256 may be formed separately from the coupler body 330 and later joined with the coupler body 330, such as by welding. In other implementations, the support body 256 may be separate from the coupler body 330 and fixed to the coupler body 330. As a non-limiting example, the support body 256 may be mechanically fastened to the coupler body 330, such as by bolting or by the mechanism depicted in U.S. Patent Publication No. 2022/0039898, entitled, “Robotic Surgical System Including a Coupler for Connecting a Tool to a Manipulator and Methods of Using the Coupler,” filed on Aug. 4, 2021, the disclosure of which is hereby incorporated herein by reference.

Moreover, the first support arm 336 and the coupler body 330 may define a first gap 306 therebetween, and the second support arm 338 and the coupler body 330 may define a second gap 308 therebetween. The first gap 306 and the second gap 308 permit the first support arm 336 and the second support arm 338 to support the support body 256, particularly the support plate 272, as well as the tracker plate 258 and the plurality of tracking elements 266, without additional and unnecessary weight which could negatively impact the performance of the surgical tracker assembly 54. Additionally, the support body 256 may further include a third support arm 340 disposed between the first support arm 336 and the second support arm 338. The third support arm 340 extends to the coupler body 330. The third support arm 340 further supports the support body 256, particularly the support plate 272, as well as the tracker plate 258 and the plurality of tracking elements 266.

The coupler 328 may be integrated into a robotic surgical system 10 for use with the localizer 44. The robotic surgical system 10 may include the coupler 328 and the robotic manipulator 14 including the plurality of links 18 and a plurality of joints J1-J6. One of the plurality of links 18 is a distal link 342 that includes a first mounting interface 344 engageable with the first coupling interface 332 of the coupler 328. The robotic surgical system 10 also includes the tool 26 including a second mounting interface 346 engageable with the second coupling interface 334 of the coupler 328. The first mounting interface 344 may have a plurality of first mounting elements 348 and the second mounting interface 346 may have a plurality of second mounting elements 350. The first coupling interface 332 may have a plurality of first coupling elements 352 arranged to align with and engage the first mounting elements 348, and the second coupling interface 334 may have a plurality of second coupling elements 354 arranged to align with and engage with second mounting elements 350 for connecting the coupler 328 to the tool 26.

The second mounting interface 346 may be engageable with the second coupling interface 334 in at least two different orientations, and optionally, in multiple orientations within a 360-degree range. The second mounting interface 346 may be engageable with the second coupling interface 334 at, or near, the joint J6. Although not required, the at least two different orientations may include a first orientation and a second orientation circumferentially spaced from the first orientation by about 90 degrees. As can be seen in FIG. 9, the support body 256 may include indicia to indicate to an operator how to orient the second coupling interface 334 relative to the second mounting interface 346 in any of the at least two different orientations, including but not limited to the first orientation and the second orientation. The surgical tracker assembly 54 can thus be oriented in a manner that provides the best line-of-sight for the plurality of tracking elements 266 to be detectable by the localizer 44.

The robotic surgical system 10 may further include a drape 356 shaped to be disposed over the robotic manipulator 14. The coupler 328 may be configured to cooperate with the drape 356 to create a sterile field barrier between the tool 26 and the robotic manipulator 14.

In a fifteenth aspect, a coupler 328 for a robotic surgical system 10 is provided. The coupler 328 includes a surgical tracker assembly 54 detectable by a localizer 44. The surgical tracker assembly 54 includes a support body 256 having an interface surface 260 defining an open receptacle 262, and a tracker plate 258 including a front side 264 supporting a plurality of tracking elements 266 detectable by the localizer 44. The tracker plate 258 also includes a rear side 268 facing away from the front side 264, with the rear side 268 including a connector 270 disposable in the open receptacle 262 to couple the tracker plate 258 to the interface surface 260 of the support body 256. The coupler 328 also includes a coupler body 330 configured to couple to the support body 256 of the surgical tracker assembly 54. In one implementation, the support body 256 is integral with the coupler body 330.

In a sixteenth aspect, a robotic surgical system 10 for use with a localizer 44 is provided. The robotic surgical system 10 includes a coupler 328. The coupler 328 includes a surgical tracker assembly 54 detectable by a localizer 44. The surgical tracker assembly 54 includes a support body 256 having an interface surface 260 defining an open receptacle 262, and a tracker plate 258 including a front side 264 supporting a plurality of tracking elements 266 detectable by the localizer 44. The tracker plate 258 also includes a rear side 268 facing away from the front side 264, with the rear side 268 including a connector 270 disposable in the open receptacle 262 to couple the tracker plate 258 to the interface surface 260 of the support body 256. The coupler 328 also includes a coupler body 330 configured to couple to the support body 256 of the surgical tracker assembly 54. The robotic surgical system 10 also includes the robotic manipulator 14 including a plurality of links 18 and a plurality of joints J1-J6. One of the plurality of links 18 is a distal link 342 that includes a first mounting interface 344 engageable with the first coupling interface 332 of the coupler 328. The robotic surgical system 10 further includes a tool 26 including a second mounting interface 346 engageable with the second coupling interface 334 of the coupler 328.

In a seventeenth aspect, the tracker plate 258 for the robotic surgical system 10 for use with the localizer 44 is provided. In this aspect, the tracker plate 258 includes the front side 264 supporting the plurality of tracking elements 266 detectable by the localizer 44. The tracker plate 258 also includes the rear side 268 facing away from the front side 264. The rear side 268 includes the first connector 270 formed as the first resilient arm 274 extending away from the rear side 268, and the first locating wall 280 rigidly affixed to the rear side 268 and disposed opposite the first connector 270. The rear side 268 also includes the second connector 282 formed as the second resilient arm 284 extending away from the rear side 268, and the second locating wall 286 rigidly affixed to the rear side 268 and disposed opposite the second connector 282.

The invention has been described in an illustrative manner, and it is to be understood that the terminology which has been used is intended to be in the nature of words of description rather than of limitation. Many modifications and variations of the present invention are possible in light of the above teachings, and the invention may be practiced otherwise than as specifically described.

What is claimed is:

1. A surgical tracker assembly for tracking an object, the surgical tracker assembly comprising:
a tracker body including a first connector; and
a mount assembly configured to couple to the tracker body, the mount assembly including a second connector configured to engage with the first connector in at least a first position and a second position different from the first position;
wherein one of the first connector and the second connector includes a multipositional receptacle extending between a first receptacle end and a second receptacle end, with the first receptacle end defining a first opening and the second receptacle end defining a second opening; and
wherein the other of the first connector and the second connector includes a projection configured to be disposed in the first opening of the first receptacle end to enable the tracker body to couple to the mount assembly in the first position and configured to be disposed in the second opening of the second receptacle end to enable the tracker body to couple to the mount assembly in the second position.

2. The surgical tracker assembly of claim 1, wherein the multipositional receptacle defines a passageway between the first receptacle end and the second receptacle end.

3. The surgical tracker assembly of claim 2, wherein the projection is configured to be disposed through the passageway such that the projection is disposed through both the first opening of the first receptacle end and the second opening of the second receptacle end.

4. The surgical tracker assembly of claim 1, wherein the first receptacle end defining the first opening and the second receptacle end defining the second opening are symmetrical with respect to one another.

5. The surgical tracker assembly of claim 1, wherein the first receptacle end defining the first opening is symmetrical about a plane bisecting the first receptacle end and the first opening.

6. The surgical tracker assembly of claim 1, wherein the multipositional receptacle is further defined as a bipositional receptacle such that the tracker body is configured to couple to the mount assembly in only the first position and the second position.

7. The surgical tracker assembly of claim 1, wherein the tracker body has a hub, a first arm extending away from the hub, a second arm extending away from the hub, and a third arm extending away from the hub, and wherein the first connector extends away from the hub transverse to the first arm, the second arm, and the third arm.

8. The surgical tracker assembly of claim 7, wherein the first arm extends a first distance from the hub, the second arm extends a second distance from the hub, and the third arm extends a third distance from the hub, wherein the first distance is greater than either of the second distance and the third distance, and wherein engagement of the first connector and the second connector orients the first arm to extend toward the object to be tracked.

9. The surgical tracker assembly of claim 7, wherein the tracker body further has a fourth arm extending away from the hub and a fifth arm extending away from the hub, and wherein the first connector extends away from the hub transverse to the first arm, the second arm, the third arm, the fourth arm, and the fifth arm.

10. The surgical tracker assembly of claim 9, wherein the first arm extends a first distance from the hub, the second arm extends a second distance from the hub, the third arm extends a third distance from the hub, the fourth arm extends a fourth distance from the hub, and the fifth arm extends a fifth distance from the hub, wherein the first distance is greater than any of the second distance, the third distance, the fourth distance, and the fifth distance, and wherein engagement of the first connector and the second connector orients the first arm to extend toward the object to be tracked.

11. The surgical tracker assembly of claim 1, wherein the first connector is plastic, and wherein the second connector is metallic.

12. The surgical tracker assembly of claim 1, wherein the projection includes a first orientation feature and the multipositional receptacle includes a second orientation feature complementary to the first orientation feature of the projection, wherein alignment of the first orientation feature and the second orientation feature orients the tracker body relative to the mount assembly in a predefined orientation during engagement of the first connector and the second connector in the first position, and wherein alignment of the first orientation feature and the second orientation feature orients the tracker body relative to the mount assembly in the predefined orientation during engagement of the first connector and the second connector in the second position.

13. The surgical tracker assembly of claim 12, wherein at least one of the first receptacle end and the second receptacle end has a keyed receptacle geometry that forms the second orientation feature, wherein the keyed receptacle geometry includes a curved portion proximal to the object to be tracked and a flat portion distal to the object to be tracked, wherein the projection has a keyed projection geometry that forms the first orientation feature, with the keyed projection geometry shaped to conform to the keyed receptacle geometry such that alignment of the keyed projection geometry and the keyed receptacle geometry orients the tracker body relative to the mount assembly in a predefined orientation, and wherein the keyed projection geometry includes a flat portion distal to the object to be tracked and a curved portion proximal to the object to be tracked.

14. The surgical tracker assembly of claim 1, wherein the projection includes a first resilient arm and a second resilient arm spaced from the first resilient arm.

15. The surgical tracker assembly of claim 14, wherein the multipositional receptacle presents an inner surface, and wherein the first resilient arm and the second resilient arm are configured to engage the inner surface of the multipositional receptacle.

16. The surgical tracker assembly of claim 14, wherein the first resilient arm includes a first lip extending away from the second resilient arm and configured to abut the multipositional receptacle to retain the tracking body to the mount assembly, and wherein the second resilient arm includes a second lip extending away from the first resilient arm and configured to abut the multipositional receptacle to retain the tracking body to the mount assembly.

17. The surgical tracker assembly of claim 16, wherein the first lip tapers toward a distal end of the first resilient arm, the second lip tapers toward a distal end of the second resilient arm, the first resilient arm includes a non-tapered portion disposed between the first lip and the distal end of the first resilient arm, and the second resilient arm includes a non-tapered portion disposed between the second lip and the distal end of the second resilient arm.

18. The surgical tracker assembly of claim 16, wherein the multipositional receptacle includes a first narrowed portion configured to align to abut the first lip of the first resilient arm and a second narrowed portion configured to align to abut the second lip of the second resilient arm.

19. The surgical tracker assembly of claim 18, wherein the first receptacle end includes a first receptacle end surface facing away from the second receptacle end, wherein the second receptacle end includes a second receptacle end surface facing away from the first receptacle end, wherein the first narrowed portion presents a first narrowed surface indented relative to one of the first receptacle end surface and the second receptacle end surface, and wherein the second narrowed portion presents a second narrowed surface indented relative to the one of the first receptacle end surface and the second receptacle end surface.

20. The surgical tracker assembly of claim 19, wherein the first narrowed portion presents a third narrowed surface indented relative to the other of the first receptacle end surface and the second receptacle end surface, and the second narrowed portion presents a fourth narrowed surface indented relative to the other of the first receptacle end surface and the second receptacle end surface.

21. The surgical tracker assembly of claim 19, wherein the multipositional receptacle presents an inner surface, wherein the first narrowed surface tapers toward the inner surface of the multipositional receptacle, and wherein the second narrowed surface tapers toward the inner surface of the multipositional receptacle.

22. The surgical tracker assembly of claim 19, wherein the first lip includes a first abutment surface shaped to correspond to the first narrowed surface, and wherein the second lip includes a second abutment surface shaped to correspond to the second narrowed surface.

23. The surgical tracker assembly of claim 14, wherein the tracker body includes a first stabilizing projection extending toward the multipositional receptacle and shaped to correspond to the multipositional receptacle, and wherein the tracker body includes a second stabilizing projection extending toward the multipositional receptacle and shaped to correspond to the multipositional receptacle.

24. The surgical tracker assembly of claim 23, wherein the first stabilizing projection and the second stabilizing projection are circumferentially offset relative to the first resilient arm and the second resilient arm.

25. The surgical tracker assembly of claim 23, wherein the first stabilizing projection and the second stabilizing projection taper toward the multipositional receptacle.

26. The surgical tracker assembly of claim 14, wherein the first resilient arm is configured to move toward the second resilient arm and the second resilient arm is configured to move toward the first resilient arm to permit the projection to be disposed in at least one of the first opening and the second opening of the multipositional receptacle.

27. The surgical tracker assembly of claim 14, wherein the first resilient arm and the second resilient arm are configured to engage the multipositional receptacle through a snap-fit.

28. The surgical tracker assembly of claim 14 further comprising a locking mechanism configured to couple to the tracker body and configured to secure the tracker body to the mount assembly.

29. The surgical tracker assembly of claim 28, wherein the locking mechanism includes a stop disposed between the first resilient arm and the second resilient arm, and wherein the stop is configured to move between a first stop position where the first resilient arm and the second resilient arm are permitted to move toward one another, and a second stop position where the first resilient arm and the second resilient arm are limited from moving toward one another.

30. The surgical tracker assembly of claim 29, wherein the stop includes a weakened region, and wherein the stop is configured to break at the weakened region.

31. The surgical tracker assembly of claim 30, wherein the weakened region has a reduced diameter relative to adjacent portions of the stop.

32. The surgical tracker assembly of claim 30, wherein the stop is configured to break at the weakened region in response to rotation of the stop from the first stop position to the second stop position, and wherein the stop is configured to remain disposed between the first resilient arm and the second resilient arm upon breaking.

33. The surgical tracker assembly of claim 1, wherein the first connector includes the projection and the second connector includes the multipositional receptacle.

34. A surgical tracker assembly comprising:
a tracker body including a first connector; and
a mount assembly including a second connector configured to releasably engage with the first connector;
wherein one of the connectors is a rigid connector and the other one of the connectors is a flexible connector configured to flex into engagement with the rigid connector; and
wherein the rigid and flexible connectors are configured to couple to one another in at least two different poses.

35. A surgical tracker assembly for tracking an object, the surgical tracker assembly comprising:
a tracker body including a first connector; and
a mount assembly configured to couple to the tracker body, the mount assembly including a second connector configured to engage with the first connector in at least a first position and a second position different from the first position;
wherein one of the first connector and the second connector is rigid; and
wherein the other of the first connector and the second connector is resilient.
